(19) United States
(12) Patent Application Publication
(10) Pub. No.: US 2023/0167171 A1
(43) Pub. Date: Jun. 1, 2023

(54) METHODS FOR TREATING ATOPIC DERMATITIS BY ADMINISTERING AN IL-4R ANTAGONIST

(71) Applicant: REGENERON PHARMACEUTICALS, INC., Tarrytown, NY (US)

(72) Inventors: Ashish BANSAL, White Plains, NY (US); Neil GRAHAM, Croton-on-Hudson, NY (US); Mohamed KAMAL, Demarest, NJ (US); Matthew P. KOSLOSKI, Yorktown Heights, NY (US); Marcella RUDDY, Wellesley, MA (US)

(21) Appl. No.: 17/821,665

(22) Filed: Aug. 23, 2022

Related U.S. Application Data
(60) Provisional application No. 63/236,035, filed on Aug. 23, 2021.
(60) Provisional application No. 63/297,908, filed on Jan. 10, 2022.
(60) Provisional application No. 63/319,500, filed on Mar. 14, 2022.
(60) Provisional application No. 63/341,948, filed on May 13, 2022.

(51) Int. Cl. C07K 16/24; A61K 45/06; A61P 17/00

(52) U.S. Cl. CPC C07K 16/247; A61K 45/06; A61P 17/00; A61K 2039/545

(57) ABSTRACT

Methods for treating moderate-to-severe atopic dermatitis in a pediatric subject are provided. In one aspect, the methods comprise administering to the subject one or more doses of an interleukin-4 receptor (IL-4R) antagonist, such as an anti-IL-4R antibody or antigen-binding fragment thereof.

### CROSS-REFERENCE TO RELATED APPLICATIONS

This application claims priority to United States Provisional Patent Application Nos. 63/236,035, filed Aug. 23, 2021, 63/297,908, filed Jan. 10, 2022, 63/319,500, filed Mar. 14, 2022, and 63/341,948, filed May 13, 2022, the contents of each of which are incorporated by reference herein.

### REFERENCE TO A SEQUENCE LISTING XML

This application contains a Sequence Listing which has been submitted electronically in XML format. The Sequence Listing XML is incorporated herein by reference. Said XML file, created on Aug. 10, 2022, is named 40848_0113USU1_SL.xml and is 236,660 bytes in size.

### FIELD OF THE INVENTION

The present disclosure relates to the use of interleukin-4 receptor (IL-4R) antagonists for treating atopic dermatitis.

### BACKGROUND

Atopic dermatitis (AD) is one of the most common skin disorders in infants and children, with onset under the age of 6 months in 45%, under the age of 1 year in 60%, and within the first 5 years in 89% of all cases (Mortz, et al., Allergy 2015, 70:836-845; Kay, et al., J Am Acad Dermatol 1994, 30:35-39). The prevalence has been estimated at 15-38% in children aged <5 years in the USA (AI-Naqeeb, et al., J Am Board Fam Med 2019, 32:191-200) and 21.5% in children aged <2 years in Germany (Illi, et al., J Allergy Clin Immunol 2004, 113:925-931). The clinical pattern of AD varies with age. Infants typically present with erythematous papules and vesicles on the cheeks, forehead, or scalp, which are exudative and intensely pruritic. The childhood phase typically occurs from 2 years of age to puberty. Children present with lichenified papules and plaques representing the more chronic disease involving the hands, feet, wrists, ankles, and antecubital and popliteal regions.

Moderate-to-severe AD markedly affects the quality of life (QoL) of both children and their families. In one study, nearly two-thirds of children with severe AD had moderately-to-highly impaired QoL (Ricci, et al., Pediatr Allergy Immunol 2007, 18:245-249). In infants, the greatest impact of AD includes itching, sleep loss, mood and behavioral changes. In children, AD disturbs sleep, increases economic costs, parental fatigue and irritability, impairs daily activities and reduces leisure and family time as well as psychological and emotional well-being. See, e.g., Ramirez, et al., JAMA Dermatol, 2019, 155:556-563.

The so-called “atopic march” in a subset of younger children, referring to the increased risk of developing asthma and/or allergic rhinitis in children with a history of AD and food allergies, suggests that AD may be an “entry point” for subsequent allergic disease. An estimated 60% of infants and young children with severe AD and 30% with mild AD develop asthma (Ricci, et al., J Am Acad Dermatol 2006, 55:765-771).

Nonpharmacological management of AD, which includes environmental control measures (e.g., avoidance of antigen and skin irritants) and skin care measures (e.g., maintaining the hydration of the skin through the use of emollients) play a supportive role, especially in children with moderate-to-severe disease. Pharmacological management of AD in children is mainly limited to topical therapy with topical corticosteroids (TCS) and topical calcineurin inhibitors (TCIs). However, long-term use of TCS in children is not recommended because of the risk of irreversible skin atrophy, dyspigmentation, acneiform eruptions, and risks associated with systemic absorption (e.g., growth retardation, hypothalamic pituitary axis effects, etc.). The use of TCI is frequently associated with skin irritation, and, a possible increased risk of malignancy (lymphoma and skin cancers) has been noted for TCIs. Moreover, neither tacrolimus nor pimecrolimus is indicated for use in children <2 years of age. Use of systemic corticosteroids is strongly discouraged in AD while other systemic immunosuppressants such as cyclosporine, methotrexate, azathioprine and mycophenolate mofetil have been used off-label despite significant potential side effects (e.g., growth retardation in children, Cushing's syndrome, hypertension, glucose intolerance, myopathy, osteonecrosis, glaucoma and cataracts). See, e.g., Lebwohl, et al/, 2019, J Drugs Dermatol, 18:122-129. Use of systemic immunosuppressants also carries the risk of rebound phenomenon, wherein symptoms of the disease may worsen significantly following cessation of treatment. Thus, there remains a high unmet medical need for an effective therapy for AD with an acceptable safety profile in infants and young children who suffer with moderate-to-severe AD.

### SUMMARY

In one aspect, method for treating atopic dermatitis (AD) or improving an AD-associated parameter in a subject are provided. In some embodiments, the method comprises administering to a subject in need thereof one or more doses of an interleukin-4 receptor (IL-4R) antagonist, wherein the subject has moderate-to-severe or severe AD and is ≥6 months to <6 years of age. In some embodiments, the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

In some embodiments, the method comprises:

(a) selecting a subject with moderate-to-severe or severe AD, wherein the subject is ≥6 months to <6 years of age; and

(b) administering to the subject one or more doses of an interleukin-4 receptor (IL-4R) antagonist, wherein the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

In some embodiments, the subject has moderate-to-severe or severe AD that is not adequately controlled by topical AD medications. In some embodiments, the subject is inadequately responsive to treatment with a topical corticosteroid (TCS) of medium or higher potency. In some embodiments, the subject is a candidate for systemic AD therapy. In some embodiments, the subject previously was administered a systemic therapy for AD. In some embodiments, the systemic AD therapy is a systemic corticosteroid. In some embodiments, the systemic AD therapy is a systemic non-steroidal immunosuppressant (such as, but not limited to, azathioprine, cyclosporine, methotrexate, or mycophenolate).

In some embodiments, the subject is aged ≥6 months to <2 years. In some embodiments, the subject is aged ≥2 to <6 years.

In some embodiments, the subject having moderate-to-severe or severe AD is 6 months, 1 year, 2 years, 3 years, 4 years or 5 years old.

In some embodiments, the subject has a baseline weight of ≥5 to <15 kg. In some embodiments, the subject is aged ≥6 months to <2 years and has a baseline weight of ≥5 to <15 kg. In some embodiments, the subject is aged ≥2 to <6 years and has a baseline weight of ≥5 to <15 kg.

In some embodiments, the subject has a baseline weight of ≥5 to <15 kg. In some embodiments, the subject is aged ≥6 months to <2 years and has a baseline weight of ≥5 to <15 kg. In some embodiments, the subject is aged ≥2 to <6 years and has a baseline weight of ≥5 to <15 kg.

In some embodiments, the subject:
(i) has a baseline Investigator's Global Assessment (IGA) score ≥3;
(ii) has a baseline Eczema Area and Severity Index (EASI) score ≥16;
(iii) has a baseline Body Surface Area (BSA) affected by AD ≥10%; and/or
(iv) has a baseline weekly average score for maximum scratch/itch intensity 4.

In some embodiments, the subject has a concurrent atopic or allergic condition selected from the group consisting of allergic rhinitis, asthma, food allergy, non-food allergy, allergic conjunctivitis, hives, chronic rhinosinusitis, nasal polyps, and eosinophilic esophagitis. In some embodiments, the subject has a food allergy.

In some embodiments, for a patient with a baseline weight of ≥5 to <15 kg, the IL-4R antagonist is subcutaneously administered at a dose of 200 mg every four weeks (Q4W); and/or for a patient with a baseline weight of ≥15 to <30 kg, the IL-4R antagonist is subcutaneously administered at a dose of 300 mg Q4W. In some embodiments, no loading dose is administered.

In some embodiments, the patient has a baseline weight ≥5 to <15 kg, and the IL-4R antagonist is subcutaneously administered an initial dose of 200 mg followed by one or more subsequent doses of 200 mg Q4W.

In some embodiments, the patient has a baseline weight 15 to <30 kg, and wherein the IL-4R antagonist is subcutaneously administered an initial dose of 300 mg followed by one or more subsequent doses of 300 mg Q4W.

In some embodiments, the IL-4R antagonist is administered for at least 16 weeks.

In some embodiments, the IL-4R antagonist is administered in combination with a topical AD medication. In some embodiments, the topical AD medication is a low-potency TCS.

In some embodiments, treatment with the IL-4R antagonist results in an increase in the number of TCS medication-free days for the subject; and/or results in a decrease in the weekly dose of TCS medication that is used by the subject.

In some embodiments, treatment with the IL-4R antagonist decreases the need for a rescue treatment (such as, but not limited to, topical corticosteroids such as medium-potency TCS or high potency TCS, systemic corticosteroids, or systemic immunosuppressants).

In some embodiments, treatment with the IL-4R antagonist results in:
a reduction from baseline in IGA score to achieve an IGA score of 0 or 1 after administration of a single dose of the IL-4R antagonist; and/or
a reduction of at least 75% from baseline in an EASI score (EASI-75) after administration of a single dose of the IL-4R antagonist.

In some embodiments, treatment with the IL-4R antagonist results in:
a reduction from baseline in IGA score to achieve an IGA score of 0 or 1 by week 16 after administration of the first dose of the IL-4R antagonist; and/or
a reduction of at least 75% from baseline in an EASI score (EASI-75) by week 16 after administration of the first dose of the IL-4R antagonist.

In some embodiments, treatment with the IL-4R antagonist results in:
a reduction of at least 50% from baseline in EASI score (EASI-50) by week 1 after administration of the first dose of the IL-4R antagonist;
a reduction of at least 75% from baseline in EASI score (EASI-75) by week 2 after administration of the first dose of the IL-4R antagonist;
a reduction of at least 90% from baseline in EASI score (EASI-90) by week 4 after administration of the first dose of the IL-4R antagonist; and/or
a ≥4-point improvement in Pruritus NRS score by week 3 after administration of the first dose of the IL-4R antagonist.

In some embodiments, treatment with the IL-4R antagonist results in an improvement in an AD-associated parameter selected from the group consisting of:
a decrease of at least 50% in EASI from baseline to Week 16 after administration of the first dose of the IL-4R antagonist;
a decrease of at least 24% in percent BSA affected by AD from baseline to Week 16 after administration of the first dose of the IL-4R antagonist;
a decrease of at least 9 points in POEM score from baseline to Week 16 after administration of the first dose of the IL-4R antagonist;
a decrease of at least 38% in SCORAD score from baseline to Week 16 after administration of the first dose of the IL-4R antagonist;
an increase of at least 1.5 points in sleep quality NRS from baseline to Week 16 after administration of the first dose of the IL-4R antagonist;
a decrease of at least 3 points in skin pain NRS from baseline to Week 16 after administration of the first dose of the IL-4R antagonist;
a decrease of at least 7 points in CDLQI score from baseline to Week 16 after administration of the first dose of the IL-4R antagonist; and a decrease of at least 8 points in IDQOL score from baseline to Week 16 after administration of the first dose of the IL-4R antagonist.

In some embodiments, treatment with the IL-4R antagonist results in a reduction in the level of one or more type 2 inflammatory biomarkers relative to a baseline value. In some embodiments, treatment with the IL-4R antagonist results in a reduction in the level of serum TARC, serum total IgE, and/or serum allergen-specific IgE in the subject relative to a baseline value, e.g., a reduction of at least 20%, 25%, 30%, 35%, 40%, 45%, 50% or more relative to a baseline value.

In some embodiments, treatment with the IL-4R antagonist prevents skin infection or reduces susceptibility to skin infection. In some embodiments, treatment with the IL-4R antagonist prevents skin bacterial infection or reduces susceptibility to skin bacterial infection (e.g., Staphylococcus).

In some embodiments, the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab.

In some embodiments, the IL-4R antagonist is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in an autoinjector. In some embodiments, the IL-4R antagonist is contained in a pen delivery device (e.g., a pre-filled pen).

In another aspect, therapeutic dosage forms of a pharmaceutical composition comprising an IL-4R antagonist are provided. In some embodiments, a therapeutic dosage form of a pharmaceutical composition comprises an IL-4R antagonist as disclosed herein (e.g., an anti-IL-4R antibody or antigen-binding fragment thereof comprising one or more CDR, HCVR, and/or LCVR sequences set forth in Table 8 below), and administration of the dosage form to a subject for at least 16 weeks provides a mean serum concentration of the IL-4R antagonist of 110 mg/L±30 mg/L (e.g., 110 mg/L±20 mg/L, 110 mg/L±15 mg/L, or 110 mg/L±10 mg/L). In some embodiments, administration of the dosage form to a subject for at least 16 weeks provides a mean serum concentration of the IL-4R antagonist of about 110 mg/L. In some embodiments, the therapeutic dose of the IL-4R antagonist is 200 mg and the dosage form is administered every four weeks. In some embodiments, the therapeutic dose of the IL-4R antagonist is 300 mg and the dosage form is administered every four weeks. In some embodiments, the subject is ≥6 months to <6 years of age.

Other embodiments will be apparent from a review of the ensuing detailed description.

### BRIEF DESCRIPTION OF THE FIGURES

FIG. 1 shows the primary endpoint of proportion of patients achieving IGA 0/1 at Week 16 with dupilumab treatment as compared to placebo. A statistically significant difference was seen starting at Week 4 and continuing through Week 16.

FIG. 2 shows the co-primary endpoint of proportion of patients achieving EASI-75 with dupilumab treatment as compared to placebo. A statistically significant difference was seen starting at Week 2 and continuing through Week 16.

FIG. 3 shows percent change from baseline in EASI over time (full analysis set) with dupilumab treatment as compared to placebo. A statistically significant improvement was seen starting at Week 1 and was sustained until Week 16.

FIGS. 4A-4C show the proportion of patients who achieved EASI-50 (FIG. 4A), EASI-75 (FIG. 4B), and EASI-90 (FIG. 4C) through Week 16 with dupilumab treatment as compared to placebo.

FIG. 5 shows percent change from baseline in pruritus NRS over time (full analysis set) with dupilumab treatment as compared to placebo. A statistically significant improvement in pruritus was seen starting at Week 1 and was sustained until Week 16.

FIG. 6 shows the proportion of patients achieving 4-point improvements in pruritus NRS score from baseline. A statistically significant increase in the proportion of responders was observed for the dupilumab treatment arm starting at Week 3 and continuing through Week 16.

FIGS. 7A and 7B show the concentrations of functional dupilumab in patients treated with dupilumab 200 mg Q4W or 300 mg Q4W. (FIG. 7A) Mean concentrations (±SD) of functional dupilumab in serum by nominal time and treatment group. (FIG. 7B) Concentrations of functional dupilumab in serum at week 16 by treatment group.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

“Atopic dermatitis” or “AD”, as used herein, means an inflammatory skin disease characterized by intense pruritus (e.g., severe itch) and by scaly and dry eczematous lesions. The term “atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present disclosure encompasses methods to treat patients with moderate-to-severe or severe AD. As used herein, “moderate-to-severe AD” is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 20% of the body's skin affected, or 10% of skin area in addition to involvement of the eyes, hands and body folds. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor. As used herein, “severe AD” is characterized by the presence of widespread skin lesions, unremitting itching, or physically or emotionally disabling disease that significantly compromises a patient's quality of life. In some cases, patients with severe AD also exhibits one or more symptoms such as excoriation, extensive skin thickening, bleeding, oozing, and/or cracking of skin, and alteration of pigmentation. In some embodiments, severe AD is refractory to treatment by a topical therapy (e.g., a topical corticosteroid, calcineurin inhibitor, or crisaborole).

As used herein, the term “subject in need thereof” refers to a human or a non-human animal having AD (e.g., moderate-to-severe AD or severe AD). In some embodiments, the term “a subject in need thereof” refers to patients with moderate-to-severe or severe AD, wherein the patient is ≥6 months and <6 years of age, e.g., a subject who is ≥6 months and <2 years of age or a subject who is ≥2 and <6 years of age. The terms “subject” and “patient” are used interchangeably herein.

In some embodiments, the term “subject in need thereof” includes patients with moderate-to-severe or severe AD who are ≥6 months and <6 years of age and who have received prior treatment with systemic therapy, or who are candidates for systemic therapy. As used herein, the term “systemic therapy” refers to systemically administered therapeutic agents (e.g., orally administered corticosteroids). The term includes systemic immunosuppressant or immunomodulatory agents. In the context of the present disclosure, the term “systemic immunosuppressant” includes, but is not limited to, cyclosporine A, methotrexate, mycophenolate mofetil, azathioprine, systemic or oral corticosteroids, Janus kinase inhibitors, and interferon-gamma. In certain embodiments, the term also includes immunobiologics such as tumor necrosis factor alpha (TNFα) inhibitors (e.g., an anti-TNFα antibody such as infliximab), CD11a inhibitors (e.g., an anti-CD11a antibody such as efalizumab), IgE inhibitors (e.g., omalizumab), CD20 inhibitors (e.g., rituximab). Systemic therapy including systemic immunosuppressants may be used for short-term treatment of flares or as a temporary measure to control disease, but their use is limited by significant side-effects, e.g., growth retardation in children, Cushing's syndrome, hypertension, glucose intolerance, myopathy, osteonecrosis, glaucoma and cataracts. Use of systemic immunosuppressants also carries the risk of rebound phenomenon, wherein symptoms of the disease may worsen significantly following cessation of treatment. In certain embodiments, the terms “systemic therapy”, “systemic therapeutic agent” and “systemic immunosuppressant” have been used interchangeably throughout this disclosure.

The term “TCS,” as used herein, includes group I, group II, group III and group IV topical corticosteroids. According to the Anatomical Therapeutic Classification System of World Health Organization, the corticosteroids are classified as weak (group 1), moderately potent (Group II) and potent (Group III) and very potent (Group IV), based on their activity as compared to hydrocortisone. Group IV TCS (very potent) are up to 600 times as potent as hydrocortisone and include clobetasol propionate and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent; also referred to interchangeably herein as “medium potency”) are 2 to 25 times as potent as hydrocortisone and include, but are not limited to, clobetasone butyrate, and triamcinolone acetonide. Group I TCS (mild; also referred to interchangeably herein as “low potency”) includes hydrocortisone.

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, methods for treating atopic dermatitis (AD) or improving an AD-associated parameter in a subject are provided. In some embodiments, the methods comprise administering to a subject having moderate-to-severe or severe AD, wherein the subject is ≥6 months and <6 years of age, one or more doses of an interleukin-4 receptor (IL-4R) antagonist. In some embodiments, the IL-4R antagonist is administered concomitantly with topical therapy for AD, such as a topical corticosteroid (TCS) or a topical nonsteroidal medication (e.g., a calcineurin inhibitor or crisaborole). In some embodiments, the IL-4R antagonist is administered concomitantly with a low-potency TCS but not a medium-potency or higher potency TCS.

In some embodiments, the subject is ≥6 months and <1 year of age. In some embodiments, the subject is ≥6 months and <2 years of age. In some embodiments, the subject is ≥1 and <2 years of age. In some embodiments, the subject is ≥2 and <4 years of age. In some embodiments, the subject is ≥4 and <6 years of age. In some embodiments, the subject is 3 and <6 years of age. In some embodiments, the subject is ≥2 and <6 years of age. In some embodiments, the subject is ≥1 and <6 years of age.

In some embodiments, a subject to be treated according to the methods disclosed herein is a subject ≥6 months and <6 years of age (e.g., a subject ≥6 months and <2 years of age or a subject ≥2 and <6 years of age) who has moderate-to-severe or severe AD that is inadequately responsive to topical therapies (e.g., TCS with or without topical calcineurin inhibitors (TCIs)) or for whom topical therapy is inadvisable (e.g., due to adverse side effects or safety risks). In some embodiments, the subject has a documented history of inadequate response to a sufficient course of outpatient treatment with topical AD medication(s). As used herein, “inadequate response” refers to a failure to achieve and maintain remission or a low disease activity state (comparable to Investigator's Global Assessment [IGA] 0=clear to 2=mild) despite treatment for at least 28 days with a topical therapy (e.g., a regimen of TCS of medium to high potency, ±TCI as appropriate). In some embodiments, a subject is considered to be “inadequately responsive to topical therapy” if the patient has received documented recent (within 6 months) systemic treatment for AD.

In some embodiments, a subject to be treated according to the methods disclosed herein is a subject ≥6 months and <6 years of age (e.g., a subject ≥6 months and <2 years of age or a subject ≥2 and <6 years of age) who has moderate-to-severe or severe AD and has previously been treated with a systemic therapy for AD.

In some embodiments, a subject to be treated has a body weight <30 kg at baseline.

In some embodiments, a subject to be treated has a body weight ≥5 kg and <30 kg at baseline.

In some embodiments, a subject to be treated has a body weight 5 kg and <15 kg at baseline.

In some embodiments, a subject to be treated has a body weight 15 kg and <30 kg at baseline.

In some embodiments, treatment with an IL-4R antagonist improves, alleviates, or reduces one or more symptoms of AD in a subject, including but not limited to pruritus, xerosis (skin dryness), eczematous lesions, erythema, papulation, edema, oozing/crusting, excoriation, lichenification, sleep disturbance, anxiety, and depression.

In some embodiments, treatment with an IL-4R antagonist improves one or more AD-associated parameters in a subject. Examples of “AD-associated parameters” include, but are not limited to: Investigators Global Assessment (IGA); Body Surface Area Involvement of Atopic Dermatitis (BSA); Eczema Area and Severity Index (EASI); SCORAD; 5-D Pruritus Scale; Pruritus Numeric Rating Scale (NRS); Patient Global Impression of Disease (PGID); Caregiver Global Impression of Disease (CGID); Patient Global Impression of Change (PGIC); Caregiver Global Impression of Change (CGIC); Children's Dermatology Life Quality Index (CDLQI); Patient Oriented Eczema Measure (POEM); Dermatitis Family Index (DFI); Patient-Reported Outcomes Measurement Information System (PROMIS) anxiety and/or depression score; and Skin Pain NRS. An “improvement in an AD-associated parameter” means an improvement (e.g., decrease) from baseline of one or more of IGA, BSA, EASI, SCORAD, 5-D Pruritus Scale, NRS/worst itch score, PGID, CGID, PGIC, CGIC, CDLQI, POEM, DFI, PROMIS, or Skin Pain NRS score. The term “baseline,” as used with respect to an AD-associated parameter, means the numerical value of the AD-associated parameter for a subject prior to or at the onset of administration of a pharmaceutical composition as disclosed herein.

To determine whether an AD-associated parameter has “improved,” the parameter is quantified at baseline and at one or more time points after administration of the pharmaceutical composition of the present disclosure. For example, an AD-associated parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular time point following initiation of treatment and the value of the parameter at baseline is used to establish whether there has been an “improvement” (e.g., a decrease) in the AD associated parameter. AD-associated parameters are described in US Patent Publication No. US 2014/0072583, incorporated herein in its entirety.

In some embodiments, an AD-associated parameter is assessed by a caregiver. In some embodiments, a parameter is quantified at baseline and at one or more time points after administration of the pharmaceutical composition based on caregiver assessment of the AD-associated parameter. In some embodiments, a caregiver reported assessment is used to assess an AD-associated parameter in a patient ≥6 months and <6 years of age, e.g., a patient ≥6 months and <4 years of age or a patient ≥6 months and <2 years of age. In some embodiments, a caregiver reported assessment is used to assess improvement in peak pruritus NRS score, global impression of disease, global impression of change, Children's Dermatology Life Quality Index (CDLQI), Patient Oriented Eczema Measure (POEM), Dermatitis Family Index (DFI) score, or Patient-Reported Outcomes Measurement Information System (PROMIS) anxiety and/or depression score. In some embodiments, improvement in itch is determined based on a caregiver reported assessment. In some embodiments, improvement in itch is assessed by caregiver reported peak pruritus NRS score. In some embodiments, improvement in pain is assessed by caregiver reported skin pain NRS score.

In some embodiments, treatment with an IL-4R antagonist according to the methods of the present disclosure results in an improvement in IGA score for the subject relative to baseline. Methods for determining an IGA score for a subject are described in the Examples section below. In some embodiments, a subject to be treated has a baseline IGA score ≥3 (e.g., an IGA score of 3 or an IGA score of 4). In some embodiments, treatment with an IL-4R antagonist results in a reduction from baseline in IGA score (e.g., from a baseline IGA score ≥3 or a baseline IGA score=4) of at least 1 point by week 3, week 4, week 8, week 12, or week 16 after administration of the first dose of the IL-4R antagonist. In some embodiments, treatment with an IL-4R antagonist results in a reduction from baseline (e.g., from an IGA score ≥3 or an IGA score=4) to an IGA score of 0 or 1 by week 3, week 4, week 8, week 12, or week 16 after administration of the first dose of the IL-4R antagonist.

In some embodiments, treatment with an IL-4R antagonist according to the methods of the present disclosure results in an improvement in an EASI score for a subject relative to baseline. Methods for determining an EASI score for a subject are described in the Examples section below. In some embodiments, a subject to be treated has a baseline EASI score of ≥16 (e.g., an EASI score ≥20, ≥25, or ≥30). In some embodiments, treatment with an IL-4R antagonist results in a reduction of at least 30%, at least 40%, at least 50%, at least 60%, at least 70%, at least 75%, at least 80%, or at least 90% from baseline in an EASI score by week 3, week 4, week 8, week 12, or week 16 after administration of the first dose of the IL-4R antagonist. In some embodiments, treatment with an IL-4R antagonist results in the subject achieving an EASI-50 response (i.e., a ≥50% improvement from baseline) by week 3, week 4, week 8, week 12, or week 16 after administration of the first dose of the IL-4R antagonist. In some embodiments, treatment with an IL-4R antagonist results in the subject achieving an EASI-75 response (i.e., a ≥75% improvement from baseline) by week 3, week 4, week 8, week 12, or week 16 after administration of the first dose of the IL-4R antagonist. In some embodiments, treatment with an IL-4R antagonist results in the subject achieving an EASI-90 response (i.e., a ≥90% improvement from baseline) by week 3, week 4, week 8, week 12, or week 16 after administration of the first dose of the IL-4R antagonist.

In some embodiments, treatment with an IL-4R antagonist improves AD signs in one or more anatomical regions (e.g., head, trunk, upper extremities, or lower extremities) or across all anatomical regions. In some embodiments, treatment with an IL-4R antagonist improves symptoms of erythema, e.g., as measured by an improvement in erythema EASI sign scores. In some embodiments, treatment with an IL-4R antagonist improves symptoms of excoriations, e.g., as measured by an improvement in excoriations EASI sign scores.

In some embodiments, treatment with an IL-4R antagonist according to the methods of the present disclosure results in an improvement in a BSA score for a subject relative to baseline. Methods for determining a BSA score for a subject are described in the Examples section below. In some embodiments, a subject to be treated has a baseline BSA score of ≥10% (e.g., ≥15%, ≥20%, ≥30%, ≥40%, ≥50%, ≥75%, or ≥90%). In some embodiments, a subject to be treated has a baseline BSA score of ≥50%. In some embodiments, treatment with an IL-4R antagonist results in a reduction of at least 10%, at least 20%, at least 30%, at least 40%, at least 50% or more from baseline in percent BSA that is affected by AD by week 3, week 4, week 8, week 12, or week 16 after administration of the first dose of the IL-4R antagonist.

In some embodiments, treatment with an IL-4R antagonist according to the methods of the present disclosure results in an improvement in a pruritus score, such as a “worst itch scale” score, also referred to herein as a Peak Pruritus Numeric Rating Scale (NRS) score, for a subject relative to baseline. Methods for determining a pruritus score are described in the Examples section below. In some embodiments, a subject to be treated has a baseline worst itch score weekly average score for maximum itch intensity that is ≥4 (e.g., ≥7). In some embodiments, treatment with an IL-4R antagonist results in a reduction of ≥3 points (e.g., ≥4 points) of a weekly average of a daily pruritus score (e.g., worst itch score) from baseline by week 3, week 4, week 8, week 12, or week 16 after administration of the first dose of the IL-4R antagonist.

In some embodiments, treatment with an IL-4R antagonist according to the methods of the present disclosure results in an improvement in a SCORAD score for the subject relative to baseline. Methods for determining a SCORAD score for a subject are described in the Examples section below. In some embodiments, a subject to be treated has a baseline SCORAD score ≥40 (e.g., a SCORAD score ≥50, ≥60, or ≥70). In some embodiments, treatment with an IL-4R antagonist results in a reduction in SCORAD score of at least 20%, at least 30%, at least 40%, at least 50%, at least 60%, at least 70%, at least 75%, at least 80%, or at least 90% from baseline by week 3, week 4, week 8, week 12, or week 16 after administration of the first dose of the IL-4R antagonist. In some embodiments, treatment with an IL-4R antagonist results in a reduction in a SCORAD component score (e.g., reduction in the SCORAD Visual Analog Scale (VAS) component score for pruritus and/or sleeplessness), e.g., a reduction in the SCORAD component score of at least 20%, at least 30%, at least 40%, at least 50%, at least 60%, at least 70%, at least 75%, at least 80%, or at least 90% from baseline by week 3, week 4, week 8, week 12, or week 16 after administration of the first dose of the IL-4R antagonist.

In some embodiments, treatment with an IL-4R antagonist according to the methods of the present disclosure results in the reduction in the level of one or more type 2 inflammatory biomarkers in the subject relative to a baseline value. In some embodiments, treatment with the IL-4R antagonist results in a reduction in the level of serum TARC, serum total IgE, and/or serum allergen-specific IgE (to allergens such as, but not limited to, food allergens [e.g., peanut, tree nut, sesame, soybeans, egg, egg white, fish, milk, crustaceans, mollusks, mustard, celery, or gluten], cat dander, dog dander, cockroach, pollen, grass, weed, dust mite [e.g., Dermatophagoides farinae or Dermatophagoides pteronyssinus], latex, medications, insects, or chemicals) in the subject relative to a baseline value. In some embodiments, treatment with an IL-4R antagonist results in a reduction in the level of one or more type 2 inflammatory biomarkers of at least 20%, 25%, 30%, 35%, 40%, 45%, 50% or more from baseline by week 3, week 4, week 8, week 12, or week 16 after administration of the first dose of the IL-4R antagonist.

In some embodiments, treatment with an IL-4R antagonist according to the methods of the present disclosure results in an improvement in skin pain, such as measured by a skin pain NRS score, for a subject relative to baseline. Methods for determining a skin pain NRS score are described in the Examples section below. In some embodiments, a subject to be treated has a baseline skin pain NRS score (e.g., skin pain NRS weekly average score) for maximum skin pain that is ≥4 (e.g., ≥7). In some embodiments, treatment with an IL-4R antagonist results in a reduction of ≥3 points (e.g., ≥4 points) of a skin pain NRS score (e.g., skin pain NRS weekly average score) from baseline by week 3, week 4, week 8, week 12, or week 16 after administration of the first dose of the IL-4R antagonist.

In some embodiments, treatment with an IL-4R antagonist reduces or eliminates the need for topical AD therapy (e.g., TCS, TCI, or crisaborole). In some embodiments, treatment with an IL-4R antagonist “reduces” the need for topical AD therapy if (1) the amount of a topical therapy for AD (e.g., TCS) that is concomitantly administered is reduced; 2) the number of days in which the topical agent (e.g., TCS) is concomitantly administered is reduced; or (3) the patient is administered a lower potency of the topical agent (e.g., the patient is switched from a medium-potency TCS to a low-potency TCS). In some embodiments, treatment with an IL-4R antagonist reduces or eliminates one or more side effects due to the topical agent (e.g., TCS). In some embodiments, treatment with an IL-4R antagonist reduces toxicity due to the topical agent (e.g., TCS). In some embodiments, the amount of the topical agent (e.g., TCS) that is concomitantly administered to the subject is decreased by at least 20%, 30%, 40%, 50%, 60%, 70%, 80% or more as compared to a baseline value for the subject or as compared to a subject that is not administered an IL-4R inhibitor. In some embodiments, treatment with an IL-4R antagonist allows for concomitant treatment with the topical agent (e.g., TCS) to be tapered off or discontinued.

In some embodiments, treatment with an IL-4R antagonist reduces the need for a rescue treatment (e.g., for AD flares, for lesions persisting or worsening under daily treatment, or for intolerable symptoms). In some embodiments, treatment with the IL-4R antagonist decreases the need for a topical rescue treatment (e.g., topical corticosteroids such as medium-potency TCS or high potency TCS). In some embodiments, treatment with the IL-4R antagonist decreases the need for a systemic rescue treatment (e.g., systemic corticosteroids or systemic immunosuppressants).

In some embodiments, treatment with an IL-4R antagonist prevents skin infection or reduces susceptibility to skin infection. In some embodiments, treatment prevents skin bacterial infection or reduces susceptibility to skin bacterial infection (e.g., Staphylococcus). In some embodiments, treatment with an IL-4R antagonist reduces the need for anti-infective medication use (e.g., antibacterials, antivirals, antifungals, or antiparasitic medications).

### Interleukin-4 Receptor Antagonists

In some embodiments, the methods of the present disclosure comprise administering to a subject in need thereof (e.g., a subject having moderate-to-severe AD who is ≥6 months and <6 years of age, such as a subject ≥6 months and <2 years of age or a subject ≥2 and <6 years of age) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein, et al., 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor, et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (K_{D}) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, N.J.). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises the amino acid sequence GFTFRDYA (SEQ ID NO:3), the HCDR2 comprises the amino acid sequence ISGSGGNT (SEQ ID NO:4), the HCDR3 comprises the amino acid sequence AKDRLSITIRPRYYGLDV (SEQ ID NO:5), the LCDR1 comprises the amino acid sequence QSLLYSIGYNY (SEQ ID NO:6), the LCDR2 comprises the amino acid sequence LGS, and the LCDR3 comprises the amino acid sequence MQALQTPYT (SEQ ID NO:8).

In some embodiments, the anti-IL-4R antibody or antigen-binding fragment thereof comprises an HCDR1 comprising the amino acid sequence GFTFRDYA (SEQ ID NO:3), an HCDR2 comprising the amino acid sequence ISGSGGNT (SEQ ID NO:4), an HCDR3 comprising the amino acid sequence AKDRLSITIRPRYYGLDV (SEQ ID NO:5), an LCDR1 comprising the amino acid sequence QSLLYSIGYNY (SEQ ID NO:6), an LCDR2 comprising the amino acid sequence LGS, and an LCDR3 comprising the amino acid sequence MQALQTPYT (SEQ ID NO:8), and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141, or International Patent Publication Nos. WO2020/096381, WO2020/239134, WO2022/052974, WO2022/136669, or WO2022/136675, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 8 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody comprises (i) an HCVR comprising the amino acid sequence of SEQ ID NO:208 (STSA-C27-VH), SEQ ID NO:209 (STSA-C27-6-33-VH), SEQ ID NO:210 (STSA-C27-7-33-VH), SEQ ID NO:211 (STSA-C27-24-56-VH), SEQ ID NO:212 (STSA-C27-47-56-VH), SEQ ID NO:213 (STSA-C27-33-33-VH), SEQ ID NO:214 (STSA-C27-56-56-VH), SEQ ID NO:215 (STSA-C27-78-78-VH), SEQ ID NO:216 (STSA-C27-82-58-VH), SEQ ID NO:217 (STSA-C27-54-54-VH), SEQ ID NO:218 (STSA-C27-36-36-VH), SEQ ID NO:219 (STSA-C27-53-53-VH), SEQ ID NO:220 (STSA-C27-67-67-VH), SEQ ID NO:221 (STSA-C27-55-55-VH), SEQ ID NO:222 (STSA-C27-59-59-VH), SEQ ID NO:223 (STSA-C27-58-58-VH), SEQ ID NO:224 (STSA-C27-52-52-VH), or SEQ ID NO:225 (STSA-C27-Y2-Y2-VH); and (ii) an LCVR comprising the amino acid sequence of SEQ ID NO:226 (STSA-C27-VL), SEQ ID NO:227 (STSA-C27-6-33-VL), SEQ ID NO:228 (STSA-C27-7-33-VL), SEQ ID NO:229 (STSA-C27-24-56-VL), SEQ ID NO:230 (STSA-C27-47-56-VL), SEQ ID NO:231 (STSA-C27-33-33-VL), SEQ ID NO:232 (STSA-C27-56-56-VL), SEQ ID NO:233 (STSA-C27-78-78-VL), SEQ ID NO:234 (STSA-C27-82-58-VL), SEQ ID NO:235 (STSA-C27-54-54-VL), SEQ ID NO:236 (STSA-C27-36-36-VL), SEQ ID NO:237 (STSA-C27-53-53-VL), SEQ ID NO:238 (STSA-C27-67-67-VL), SEQ ID NO:239 (STSA-C27-55-55-VL), SEQ ID NO:240 (STSA-C27-59-59-VL), SEQ ID NO:241 (STSA-C27-58-58-VL), SEQ ID NO:242 (STSA-C27-52-52-VL), or SEQ ID NO:243 (STSA-C27-Y2-Y2-VL).

In some embodiments, an anti-IL-4Rα antibody used in the methods of the present disclosure can have pH-dependent binding characteristics. For example, an anti-IL-4Rα antibody for use as disclosed herein may exhibit reduced binding to IL-4Rα at acidic pH as compared to neutral pH. Alternatively, an anti-IL-4Rα antibody for use as disclosed herein may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2 and HCDR3) contained within a heavy chain variable region (HCVR) having an amino acid sequence of SEQ ID NO:1, and the three light chain CDRs (LCVR1, LCVR2, and LCVR3) contained within a light chain variable region (LCVR) having an amino acid sequence of SEQ ID NO:2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); AI-Lazikani, et al., J. Mol. Biol. 273:927-948 (1997); and Martin, et al., Proc. Natl. Acad. Sci. USA 86:9268-9272 (1989). Public databases are also available for identifying CDR sequences within an antibody.

### Pharmaceutical Compositions

In one aspect, the present disclosure provides methods that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, Pa. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti, et al., 1991, Pharmaceut. Res. 8:1351). Specific exemplary doses of anti-IL4R antibodies, and administration regimens involving the same, that can be used in the context of the present disclosure are disclosed elsewhere herein.

In some embodiments, an IL-4R antagonist or a pharmaceutical composition of the present disclosure is contained within a container. Thus, in another aspect, containers comprising an IL-4R antagonist or a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ 1, and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem. 262:4429-4432).

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present disclosure are disclosed, e.g., in U.S. Pat. No. 8,945,559.

### Dosage and Administration

In some embodiments, an IL-4R antagonist (e.g., anti-IL-4R antibody) is administered to a subject (e.g., a subject ≥6 months and <6 years of age) according to the methods of the present disclosure in a therapeutically effective amount. As used herein with reference to an IL-4R antagonist, the phrase “therapeutically effective amount” means an amount of IL-4R antagonist that results in one or more of: (a) an improvement in one or more AD-associated parameters (as mentioned elsewhere herein); and/or (b) a detectable improvement in one or more symptoms or indicia of atopic dermatitis.

In the case of an anti-IL-4R antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In some embodiments, a therapeutically effective amount is from about 50 mg to about 600 mg, or from about 100 mg to about 600 mg, or from about 200 mg to about 600 mg. In certain embodiments, 50 mg, 75 mg, 100 mg, 125 mg, 150 mg, 200 mg, 250 mg, or 300 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the methods disclosed herein comprise administering an IL-4R antagonist to a subject once a month or twice a month.

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”).

In some embodiments, the initial or loading dose and the one or more secondary or maintenance doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In some embodiments, one or more maintenance doses of the IL-4R antagonist are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 week, 2 weeks, 3 weeks, or 4 weeks after the immediately preceding dose. Similarly, in some embodiments involving multiple tertiary doses, each tertiary dose is administered at the same frequency as the other tertiary doses. For example, each tertiary dose may be administered to the patient 1 week, 2 weeks, 3 weeks, or 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, a therapeutically effective amount of an IL-4R antagonist (e.g., anti-IL-4R antibody) comprises 200 mg administered every four weeks (Q4W).

In some embodiments, a therapeutically effective amount of an IL-4R antagonist (e.g., anti-IL-4R antibody) comprises 300 mg administered every four weeks (Q4W).

In some embodiments, for a subject having moderate-to-severe or severe AD who is ≥6 months to <6 years of age, a therapeutically effective amount of an IL-4R antagonist (e.g., anti-IL-4R antibody) comprises 200 mg administered every four weeks (Q4W), if the subject is ≥5 to <15 kg in weight.

In some embodiments, for a subject ≥5 to <15 kg in weight, the IL-4R antagonist (e.g., anti-IL-4R antibody) is administered at a dose of 200 mg on day 1 and then 200 mg Q4W starting 4 weeks later (i.e., 4 weeks after the day 1 dose).

In some embodiments, for a subject having moderate-to-severe or severe AD who is ≥6 months to <6 years of age, a therapeutically effective amount of an IL-4R antagonist (e.g., anti-IL-4R antibody) comprises 300 mg administered every four weeks (Q4W), if the subject is ≥15 to <30 kg in weight.

In some embodiments, for a subject ≥15 to <30 kg in weight, the IL-4R antagonist (e.g., anti-IL-4R antibody) is administered at a dose of 300 mg on day 1 and then 300 mg Q4W starting 4 weeks later (i.e., 4 weeks after the day 1 dose).

### Combination Therapies

In some embodiments, the methods of the present disclosure comprise administering to the subject (e.g., a subject ≥6 months and <6 years of age) an IL-4R antagonist according to the disclosure (e.g., an anti-IL-4R antibody) in combination with one or more additional therapeutic agents. In some embodiments, the additional therapeutic agent is a topical therapeutic agent, e.g., a TCS or a topical nonsteroidal medication such as a TCI or crisaborole. As used herein, the expression “in combination with” means that the topical therapy (e.g., TCS) is administered before, after, or concurrent with the IL-4R inhibitor. The term “in combination with” also includes sequential or concomitant administration of IL-4R inhibitor and the topical therapy (e.g., TCS).

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than about 10 minutes (before, after, or at the same time) of administration of the pharmaceutical composition comprising the IL-4R antagonist, or administered to the subject as a single combined dosage formulation comprising both the additional therapeutic agent and the IL-4R antagonist.

In some embodiments, the additional therapeutic agent is a TCS. In some embodiments, the TCS is a medium-potency TCS. In some embodiments, the TCS is a low-potency TCS. In some embodiments, the additional therapeutic agent is a TCI. In some embodiments, the additional therapeutic agent is crisaborole.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial Investigating the Pharmacokinetics, Efficacy, and Safety of Dupilumab in Children Aged 6 Months to <6 Years with Moderate-to-Severe Atopic Dermatitis

### Study Design and Objectives

This study was a two-part (parts A and B) Phase 2/3 study (LIBERTY AD PRE-SCHOOL; NCT03346434) to evaluate the safety, PK, and efficacy of dupilumab in patients 6 months to less than 6 years of age with moderate-to-severe AD. Part A was an open-label, single-ascending dose, sequential cohort phase 2 study in patients ≥6 months to <6 years of age with severe AD. The primary objective of Part A was to obtain safety and PK data in this patient population to guide dose selection for Part B. The results from Part A are described in PCT/US2021/024419, incorporated by reference herein.

Part B was a randomized, double-blind, parallel-group, placebo-controlled study in which study treatments are administered concomitantly with topical therapy to patients. The primary objective of Part B was to demonstrate efficacy of dupilumab in combination with prescription topical therapy in pediatric patients, 6 months to less than 6 years of age, suffering from moderate-to-severe AD. As the efficacy and safety of dupilumab for the treatment of AD had not been established in patients ≥6 months to <6 years, a placebo control was a scientifically essential element of the study design to enable adequate assessment and interpretation of the treatment effect and safety profile. It is particularly relevant for pediatric patients, in whom spontaneous remission of AD over time has been described. Patients completing the treatment period of part B (week 16) were offered an opportunity to screen for the OLE study at end-of-treatment (EOT) visit.

Dupilumab is a fully human anti-IL-4R antibody comprising a heavy chain comprising the amino acid sequence of SEQ ID NO:9 and a light chain comprising the amino acid sequence of SEQ ID NO:10; an HCVR/LCVR amino acid sequence pair comprising SEQ ID NOs:1/2; and heavy and light chain CDR sequences comprising SEQ ID NOs:3-8.

This study was conducted in accordance with the provisions of the Declaration of Helsinki, the International Conference on Harmonization Good Clinical Practices guideline, and applicable regulatory requirements. The protocol was reviewed and approved by institutional review boards/ethics committees at all sites. For all patients, written informed consent was obtained from a parent or legal guardian.

### Patient Population

For Part B, the study population included pediatric patients (aged ≥6 months to <6 years at the time of screening visit) having moderate-to-severe AD that cannot be adequately controlled with topical AD medications.

Inclusion Criteria: A patient had to meet the following criteria to be eligible for inclusion in the study: (1) male or female, ≥6 months to <6 years of age at time of screening visit; (2) diagnosis of AD according to the American Academy of Dermatology consensus criteria (Eichenfield 2003) at screening visit; (3) documented recent history (within 6 months before the screening visit) of inadequate response to topical AD medication(s); (4) IGA ≥3 at screening and baseline visits; (5) EASI 16 at the screening and baseline visits; (6) BSA 10% at screening and baseline visits; (7) have applied a stable dose of topical emollient (moisturizer) twice daily for at least the 7 consecutive days immediately before the baseline visit; (8) parent or legal guardians, as appropriate, are able to understand and complete the study requirements and study-related questionnaires; (9) parents/caregiver or legal guardians, as appropriate, are able to understand and complete the study requirements and study-related questionnaires; (10) baseline worst scratch/itch score weekly average score for maximum scratch/itch intensity ≥4; (11) at least 11 (of a total of 14) daily applications of low potency TCS during the 2-week TCS standardization period (beginning on day −14) leading up to the baseline visit.

NOTE for inclusion criteria (3): Patients who are unable to achieve and/or maintain remission and low disease activity (an IGA score of less than 3) despite treatment with a daily regimen of medium to higher potency TCS (±TCI as appropriate), applied for ≥28 days of use, or for the maximum duration recommended by the product prescribing information, whichever is shorter, will meet the definition of inadequate response for the purpose of this study. Patients with documented systemic treatment for AD in the past 6 months are also considered as inadequate responders to topical treatments and are potentially eligible for treatment with dupilumab after appropriate washout. Acceptable documentation includes contemporaneous chart notes that record topical medication prescription and treatment outcome, or investigator documentation based on communication with the patient's treating physician. If documentation is inadequate, potential patients may be offered a course of treatment with a daily regimen of TCS of medium or higher potency (±TCI as appropriate), applied for at least 28 days during the screening period, or for the maximum duration recommended by the product prescribing information, whichever is shorter. Patients who demonstrate inadequate response during this period, as defined above, will still be eligible for inclusion in the study.

NOTE for inclusion criteria (10): Baseline worst scratch/itch average score for maximum itch intensity will be determined based on the average of daily worst scratch/itch NRS scores for maximum scratch/itch intensity (the daily score ranges from 0 to 10) during the 7 days immediately preceding randomization (does not include the day of randomization). A minimum of 4 daily scores out of the 7 days is required to calculate the baseline average score. A daily score consists of answers to the following question: “How would you rate your child's scratching/itching at its worst in the past 24 hours?” For patients who do not have at least 4 daily scores reported during the 7 days immediately preceding the planned randomization date, randomization should be postponed until this requirement is met, but without exceeding the 56-day maximum duration for screening.

Exclusion Criteria: The following were exclusion criteria for the study: (1) participation in a prior dupilumab clinical study; (2) history of important side effects of low potency topical corticosteroids (e.g., intolerance to treatment, hypersensitivity reactions, significant skin atrophy, systemic effects), as assessed by the investigator or patient's treating physician; (3) treatment with a topical investigational drug within 2 weeks or within 5 half-lives (if known), whichever is longer, or treatment with a systemic investigational drug prior to the baseline visit; (4) treatment with a TCI within 2 weeks prior to the baseline visit; (5) having used any of the following treatments within 4 weeks before the baseline visit, or within a period equal to 5 times the half-life of the drug, before the baseline visit, whichever is longer: (a) immunosuppressive/immunomodulating drugs (e.g., systemic corticosteroids, cyclosporine, mycophenolate-mofetil, interferon gamma, Janus kinase inhibitors, azathioprine, methotrexate, etc.); (b) phototherapy for AD; (6) treatment with biologics, as follows: (a) any cell-depleting agents including but not limited to rituximab: within 6 months before the baseline visit, or until lymphocyte and CD 19+ lymphocyte count returns to normal, whichever is longer; (b) other biologics: within 5 half-lives (if known) or 16 weeks before the baseline visit, whichever is longer; (7) Treatment with crisaborole within 2 weeks prior to the baseline visit; (8) Treatment with a live (attenuated) vaccine within 4 weeks before the baseline visit; (9) planned or anticipated use of any prohibited medications and procedures during study treatment; (10) initiation of treatment of AD with prescription moisturizers or moisturizers containing additives such as ceramide, hyaluronic acid, urea, or filaggrin degradation products during the screening period (patients may continue using stable doses of such moisturizers if initiated before the screening visit); (11) active chronic or acute infection requiring treatment with systemic antibiotics, antivirals, antiprotozoals, or antifungals within 2 weeks before the baseline visit. [Note: patients may be rescreened after infection resolves. A patient with mild, localized superficial infection can be included in the study based on investigator discretion.]; (12) established diagnosis of a primary immunodeficiency disorder (e.g., severe combined immunodeficiency, Wiskott Aldrich Syndrome, DiGeorge Syndrome, X-linked Agammaglobulinemia, common variable immunodeficiency), or secondary immunodeficiency. Patients suspected to have immunodeficiency based on their clinical presentation (history of invasive opportunistic infections e.g. tuberculosis, histoplasmosis, listeriosis, coccidioidomycosis, pneumocystosis, chronic mucocutaneous candidiasis, etc., or otherwise recurrent infections of abnormal frequency or prolonged duration suggesting an immune compromised status, as judged by the investigator) will also be excluded from the study; (13) eczema as part of a genodermatosis syndrome like Netherton's syndrome, Hyper IgE syndrome, Wiskott-Aldrich Syndrome, etc.; (14) known history of human immunodeficiency virus (HIV) infection or HIV seropositivity at the screening visit; (15) established diagnosis of hepatitis B viral infection at the time of screening or is positive for hepatitis B surface antigen (HBsAg) or hepatitis B core antibody (HBcAb) at the time of screening. [Note: Patients who are HBsAg negative and HBsAb positive are considered immune after a natural infection has cleared or they have been vaccinated against hepatitis B. Therefore, they are acceptable for the study. These patients will be allowed to enroll into the study, but will be followed using routine clinical and liver function tests.]; (16) established diagnosis of hepatitis C viral infection at the time of screening or is positive for hepatitis C antibody at the screening visit; (17) history of past or current tuberculosis or other mycobacterial infection; (18) have known hepatic disease or are on current treatment for hepatic disease including but not limited to acute or chronic hepatitis, cirrhosis, or hepatic failure, or has evidence of liver disease as indicated by persistent (confirmed by repeated tests ≥2 weeks apart) elevated transaminases (alanine aminotransferase [ALT] and/or aspartate aminotransferase [AST]) >3 times the upper limit of normal (ULN) during the screening period; (19) presence of any one or more of the following abnormalities in laboratory test results at screening: (i) Platelets ≤100×10³/μL; (ii) Neutrophils ≤1.0×10³/μL for patients <1 year of age; neutrophils ≤1.5×10³/μL for patients 1 year to <6 years of age; (iii) Eosinophils >5000/μL; (iv) Creatine phosphokinase (CPK) >5×ULN; (v) Serum creatinine >1.5×ULN. [Note: If an abnormal value is detected at screening, a repeat test should be performed to confirm the abnormality. If the repeat test confirms the abnormality, the patient will be categorized as a screen failure.]; (20) presence of skin comorbidities that may interfere with study assessments, including but not limited to conditions like scabies, seborrheic dermatitis, cutaneous T cell lymphoma, psoriasis, etc.; (21) history of malignancy before the baseline visit; (22) diagnosed active endoparasitic infections; suspected or high risk of endoparasitic infection, unless clinical and (if necessary) laboratory assessment have ruled out active infection before randomization; (23) severe concomitant illness(es) that, in the investigator's judgment, would adversely affect the patient's participation in the study. Examples include, but are not limited to patients with short life expectancy, patients with major congenital malformations, patients with cardiovascular conditions (e.g. major, clinically significant congenital cardiovascular abnormalities), severe renal conditions, hepato-biliary conditions (e.g. Child-Pugh class B or C), active major autoimmune diseases (e.g. lupus, inflammatory bowel disease etc.), other severe endocrinological, gastrointestinal, metabolic, pulmonary, neurological or lymphatic diseases. The specific justification for patients excluded under this criterion will be noted in study documents (chart notes, case report forms [CRF], etc.). (24) any other medical or psychological condition including relevant laboratory abnormalities at screening that, in the opinion of the investigator, suggest a new and/or insufficiently understood disease, may present an unreasonable risk to the study patient as a result of his/her participation in this clinical trial, may make patient's participation unreliable, or may interfere with study assessments; (25) planned major surgical procedure during the patient's participation in this study; (26) patient or his/her immediate family is a member of the dupilumab investigational team; (27) body weight <5 kg or ≥30 kg at baseline.

### Study Treatments

The dupilumab treatment group in Part B of the current study was a weight-tiered fixed-dose regimen of 200 mg Q4W SC for children with a baseline body weight ≥5 to <15 kg and 300 mg Q4W SC for children with a baseline body weight 15 to <30 kg. The doses selected for part B of the study were based on PK data gathered from Part A (patients ≥2 years to <6 years of age or ≥6 months to <2 years of age) and integrated in a population PK model for pediatric patients. PK parameters, like clearance (CL) and volume of distribution (V), do not scale linearly with body weight for younger children (Zhang, et al., J Clin Pharmacol. 2015; 55:S103-S115). Because the reductions of CL and V in children are less than weight-proportional, a weight-normalized dose directly scaled down from an adult dose is likely to yield sub-optimal exposure in children.

Study drug treatments for Part B were as follows:
Dupilumab 175 mg/mL: Each 1.14 mL single-use, prefilled glass syringe with snap-off cap delivers 200 mg of study drug (1.14 mL of a 175 mg/mL solution).
Dupilumab 150 mg/mL: Each 2.25 mL single-use, prefilled glass syringe with snap-off cap delivers 300 mg of study drug (2.0 mL of a 150 mg/mL solution).
Placebo-matching dupilumab was prepared in the same formulation without the addition of protein (i.e., active substance, anti-IL-4Rα mAb). Two matching placebo formulations were used:
   1.14 mL placebo matching 200 mg dupilumab formulation
   2 mL placebo matching 300 mg dupilumab formulation

SC injection sites of the study drug should be alternated among the different quadrants of the abdomen (avoiding navel and waist areas), upper thighs, and upper arms so that the same site is not injected for 2 consecutive injections. To allow for adequate assessment of possible injection site reactions, study drug should be administered only into areas of normal looking skin (for patients with 100% BSA involvement, the injection should be administered into as near normal looking skin as possible).

In Part B, all patients were required to have moisturizers (emollients) applied at least twice daily for at least the 7 consecutive days immediately before randomization (does not include day of randomization). At least 11 of the 14 total applications must be applied for the patient to remain eligible for the study. Patients were to continue to have moisturizers applied throughout the remainder of the study (all 28 weeks where applicable). However, to allow adequate assessment of skin dryness, moisturizers were not applied for at least 8 hours before each clinic visit. All types of moisturizers were permitted, but patients could not initiate treatment with prescription moisturizers or moisturizers containing additives during the screening period or during the study. Patients could continue using stable doses of such moisturizers if initiated before the screening visit.

Starting on day −14, all patients in part B were required to initiate treatment with TCS using a standardized regimen according to the following guidelines:
Apply low potency TCS once daily to areas with active lesions. Based on investigator discretion, low/mild potency TCS may also be used on areas of thin skin (face, neck, intertriginous, and genital areas, areas of skin atrophy, etc.).
Once the patient achieves an IGA score of 2 or less, lower frequency of use of low potency TCS to 3 times per week, and then stop once lesions are clear (IGA=0). Patients should be instructed to only use TCS on active lesions, and to stop use of TCS if lesions clear completely in between clinic visits.
If lesions return, reinstitute treatment with low potency TCS, with the same step-down approach described above upon lesion resolution.
For lesions persisting or worsening under daily treatment with low potency TCS, patients may be treated (rescued) with medium or high potency TCS (super potent TCS are not allowed to be used, even as rescue). The use of medium or high potency TCS as rescue is only allowed after day 14. A medium or high potency TCS should normally be restricted to use on non-delicate skin sites (excluding face, flexures, groin), and should not be used for a prolonged period in order to prevent the development of cutaneous atrophy and adrenal axis suppression. Low potency steroids should normally be used for delicate skin sites (face, flexures, groin) during these flares. Topical calcineurin inhibitors may be used for rescue, alone or in combination with TCS, but the use of TCIs should be reserved for problem areas only (e.g., face, neck, intertriginous, and genital areas, etc.).

### Outcomes Assessed

The primary endpoint for Part B of the study was the proportion of patients with an IGA score of 0 to 1 (on a 5-point scale) at week 16. The co-primary endpoints of the study (for EU and EU Reference Market Countries) were the proportion of patients with EASI-75 (≥75% improvement from baseline) at week 16, and the proportion of patients with an IGA score of either 0 or 1 (on a 5-point scale) at week 16.

The key secondary endpoints in Part B were: EASI-75 (≥75% improvement from baseline) at week 16 (not applicable for EU and EU Reference Market Countries); percent change in EASI score from baseline to week 16; and percent change from baseline to week 16 in weekly average of daily worst scratch/itch NRS score.

Other secondary endpoints in Part B were: proportion of patients with EASI-50 at week 16; proportion of patients with EASI-90 at week 16; change from baseline to week 16 in percent BSA affected by AD; percent change from baseline to week 16 in SCORAD; change from baseline to week 16 in weekly average of daily worst scratch/itch NRS score; proportion of patients with improvement (reduction) of weekly average of daily worst scratch/itch NRS score ≥4 from baseline at week 16; proportion of patients with improvement (reduction) of weekly average of daily worst scratch/itch NRS score ≥3 from baseline at week 16; change from baseline to week 16 in skin pain NRS; change from baseline to week 16 in sleep quality NRS; change from baseline to week 16 in health-related quality of life, as measured by CDLQI (patients 4 years of age) and IDQOL (patients <4 years of age); change from baseline to week 16 in DFI; change from baseline to week 16 in POEM; topical treatment for AD—proportion of TCS medication-free days from baseline to week 16; mean weekly dose of low potency TCS through week 16; mean of caregiver missed workdays from baseline to week 16; incidence of skin infection TEAEs (excluding herpetic infections) through week 16; and incidence of SAEs through week 16.

Procedures for assessing efficacy (e.g., using IGA, EASI, SCORAD, BSA, NRS, or other methods of assessment) are described below and are also described in WO 2018/057776, incorporated by reference herein.

Investigator's Global Assessment: The IGA is an assessment instrument used in clinical studies to rate the severity of AD globally, based on a 5-point scale ranging from 0 (clear) to 4 (severe). The IGA score is assessed at screening, baseline, and on specified days during and/or after treatment.

Eczema Area and Severity Index: The EASI is a validated measure used in clinical practice and clinical trials to assess the severity and extent of AD (Hanifin, et al., 2001, Exp. Dermatol. 10: 11-18). The EASI is a composite index with scores ranging from 0 to 72. Four AD disease characteristics (erythema, thickness [induration, papulation, edema], scratching [excoriation], and lichenification) each are assessed for severity by the investigator or designee on a scale of “0” (absent) through “3” (severe). In addition, the area of AD involvement is assessed as a percentage by body area of head, trunk, upper limbs, and lower limbs, and converted to a score of 0 to 6. In each body region, the area is expressed as 0, 1 (1% to 9%), 2 (10% to 29%), 3 (30% to 49%), 4 (50% to 69%), 5 (70% to 89%), or 6 (90% to 100%). The EASI score is assessed at screening, baseline, and on specified days during and/or after treatment.

SCORing Atopic Dermatitis: The SCORing Atopic Dermatitis (SCORAD) is a validated tool used in clinical research and clinical practice that was developed to standardize the evaluation of the extent and severity of AD (European Task Force on Atopic Dermatitis 1993, Dermatol. 186: 23-31). There are 3 components to the assessment: A=extent or affected BSA, B=severity, and C=subjective symptoms. The extent of AD is assessed as a percentage of each defined body area and reported as the sum of all areas, with a maximum score of 100% (assigned as “A” in the overall SCORAD calculation). The severity of 6 specific symptoms of AD (redness, swelling, oozing/crusting, excoriation, skin thickening/lichenification, and dryness) is assessed using the following scale: none (0), mild (1), moderate (2), or severe (3) (for a maximum of 18 total points, assigned as “B” in the overall SCORAD calculation). Subjective assessment of pruritus/itch and sleeplessness is recorded for each symptom by the patient or relative on a Visual Analogue Scale, where 0 is no itch (or sleeplessness) and 10 is the worst imaginable itch (or sleeplessness), with a maximum possible score of 20. This parameter is assigned as “C” in the overall SCORAD calculation. The SCORAD is calculated as: A/5+7B/2+C where the maximum is 103. The SCORAD score is assessed at screening, baseline, and on specified days during and/or after treatment.

Body Surface Area Involvement of Atopic Dermatitis: Body surface area (BSA) affected by AD is assessed for each section of the body using the rule of nines (the possible highest score for each region is: head and neck [9%], anterior trunk [18%], back [18%], upper limbs [18%], lower limbs [36%], and genitals [1%]) and is reported as a percentage of all major body sections combined. BSA is assessed at screening, baseline, and on specified days during and/or after treatment.

Peak Pruritus Numeric Rating Scale: Peak Pruritus Numeric Rating Scale (NRS) is a validated patient-reported measure for evaluating worst itch intensity (Yosipovitch, et al., Br J Dermatol, 2019, 181:761-769). This is an 11-point scale (0 to 10), in which 0 indicates no itching while 10 indicates worst itching possible, in which the patient (or caregiver) assesses the intensity of peak (worst) pruritus (itch) during the past 24 hours. The parents/caregivers are asked to answer, based on what they observe and what the child tells them (if applicable), “How would you rate your child's scratching/itching at its worst in the past 24 hours?” Pruritus is assessed by the parent/caregiver on a daily basis using an e-diary throughout the entire study.

Assessment of skin pain: Skin pain is assessed by the parent/caregiver at specified time points using a skin pain NRS that was developed and tested for the study-relevant age group. This is an 11-point scale (0 to 10) in which 0 indicates no pain while 10 indicates worst pain possible. The parents/caregivers are asked to: “Think about all the areas of your child's skin with eczema. Answer the question “How would you rate your child's skin pain at its worst in the past 24 hours?”, based on what you observe and what your child tells you (if applicable).”

Sleep quality and other sleep-related concepts: A sleep diary is completed by the parent/caregiver at specified time points. The sleep diary includes 2 questions assessing the caregiver's sleep, and 6 questions assessing the child's sleep based on caregiver observation. Sleep diary items, either alone or in combination will serve as subjective measures of sleep quality, difficulty falling asleep, nighttime awakenings, and sleep duration. Sleep quality is measured using an 11-point NRS (0 to 10) in which 0 indicates worst possible sleep while 10 indicates best possible sleep. The parents/caregivers are instructed to complete the questions about the child's sleep upon awakening for the day.

Caregiver Global Impression of Disease: The CGID is an assessment instrument used by the parent/caregiver in clinical studies to rate their child's eczema symptoms during the past 7 days. An appropriate version of the CGID was being developed and tested for the study-relevant age group. Parents/caregivers rate their child's disease based on the 5-level scale (no symptoms, mild, moderate, severe, very severe) as follows: “Overall, how would you rate your child's eczema symptoms during the past 7 days?”

Caregiver Global Impression of Change: The CGIC is a caregiver administered tool that is currently being developed and tested for the study-relevant age group, used to measure change in the child's eczema symptoms (e.g., to compare the child's eczema symptoms from the beginning of the study to a later timepoint when the CGIC assessment is completed). Parents/caregivers respond to the following question based on the 7-level scale (much better, moderately better, a little better, no change, a little worse, moderately worse, much worse) as follows: “Compared to before your child started the study, how would you rate his or her eczema now?”

Children's′ Dermatology Life Quality Index (CDLQI): The CDLQI is a validated questionnaire designed to measure the impact of skin disease on the QOL in children ≥4 years of age (Lewis-Jones 1995). The aim of the questionnaire is to measure how much a patient's skin problem has affected the patient over a recall period of the past week. The CDLQI is assessed at specified time points during the study.

To complete the questionnaire, patients need to provide responses to 10 questions which focus on domains such as symptoms feelings associated with disease, the impact of the disease on leisure, school or holidays, personal relationships, sleep, and side effects of treatment for the skin disease. The instrument has a recall period of 7 days. Nine of the 10 questions are scored as follows: very much=3, quite a lot=2, only a little=1, not at all=0, question unanswered=0. One question has an additional possible response (prevented school), which is assigned a score of 3. The CDLQI for a patient is the sum of the score of each question with a maximum of 30 and a minimum of 0. The higher the score, the greater the impact is on the QOL. The CDLQI can also be expressed as a percentage of the maximum possible score of 30.

A cartoon version of the CDLQI is administered to patients 4 to 5 years of age, with the assistance of a parent or adult “as necessary”. If assistance of parent or adult caregiver is required, it is recommended that the same person assist the patient throughout the study. The cartoon version of the CDLQI uses the same text and scoring system as the original CDLQI but includes 10 color drawings of a dog illustrating the theme of each question.

Infants' Dermatology Quality of Life Index (IDQOL): The IDQOL is a validated questionnaire developed to measure the impact of skin disease on the QOL of infants and preschool children <4 years of age (Lewis-Jones 2001). The IDQOL is to be completed by the child's parent or caregiver. It is recommended that the same person complete the questionnaire on behalf of the patient throughout the study. The questionnaire consists of 10 questions related to itching and scratching; mood of the child; how long it takes for the child to get to sleep; whether the eczema has interfered with the child's playing, swimming or participation in other family activities; problems during mealtimes; problems caused by treatment; level of comfort while dressing or undressing the child; and problems during bathing. Each question asks about the impact over the previous week and is scored on a scale of 0 (minimum impact) to 3 (maximum impact). The IDQOL for a patient is the sum of the score of each question with a maximum of 30 and a minimum of 0. The higher the score, the greater the impact is on the QOL. The IDQOL can also be expressed as a percentage of the maximum possible score of 30. The IDQOL is assessed at specified time points during the study.

Dermatitis Family Index: The impact on family life has been documented in families of children with very severe AD. The DFI was the first instrument assessing the impact of having a child with AD on family QOL (Lawson 1998). The 10-item disease specific questionnaire was formed after ethnographical interviews and focus groups revealed the areas of family QOL affected by AD. The self-administered instrument is completed by an adult family member of a child affected by dermatitis. It is recommended that the same person complete the questionnaire on behalf of the patient throughout the study. The items inquire about housework, food preparation, sleep, family leisure activity, shopping, expenditure, tiredness, emotional distress, relationships and the impact of helping with treatment on the primary caregiver's life. The DFI questions are scored on a four-point Likert scale ranging from 0 to 3, so that the total DFI score ranges from 0 to 30. The time frame of reference is the past week. A higher DFI score indicates greater impairment in family QOL as affected by AD. The DFI is assessed at specified time points during the study.

Patient-Oriented Eczema Measure (POEM): The POEM is a 7-item, validated questionnaire used in clinical practice and clinical trials to assess disease symptoms in children and adults (Charman 2004). The format is a response to 7 items (dryness, itching, flaking, cracking, sleep loss, bleeding, and weeping) based on frequency of these disease symptoms during the past week (i.e., 0=no days, 1=1 to 2 days, 2=3 to 4 days, 3=5 to 6 days, and 4=all days) with a scoring system of 0 to 28; the total score reflects disease-related morbidity. The POEM is assessed at specified time points during the study.

### Pharmacokinetic Analysis

Functional dupilumab concentrations in serum were analyzed using a validated enzyme-linked immunosorbent assay (ELISA) as previously described. The lower limit of quantitation (LLoQ) for dupilumab in undiluted human serum is 0.0780 mg/L. Serum for PK analyses was collected at baseline (before dupilumab injection) and on study days 3, 8, 18, and 29.

PK parameters, including maximum concentration (Cₘₐₓ), dose-normalized Cₘₐₓ (Cₘₐₓ/Dose), time to maximum concentration (tₘₐₓ), last observed concentration (Cₗₐₛₜ), time to last observed concentration (tₗₐₛₜ), area under the curve (AUC) from time zero to the last observed concentration (AUCₗₐₛₜ), and dose-normalized AUClast (AUCₗₐₛₜ/Dose), were determined using non-compartmental methods and actual sampling times. Mean concentration-time profiles are presented using nominal sampling times.

### Biomarker Analysis

Thymus and activation-regulated chemokine (TARC) and total serum IgE are markers of Th2 activity as downstream mediators in the IL-4/IL-13 signaling pathway. These analytes were assessed as measures of Th2 activity and PD effect of dupilumab. TARC levels have also been closely associated with AD disease activity and severity (Beck, et al., New Engl J Med 2014, 371:130-139) and were evaluated as an exploratory marker of efficacy. Serum samples for measurements of biomarkers (including TARC, total IgE, immunoglobin profiling, antigen-specific IgE, and LDH) were collected at specified time points. Methods for measuring serum TARC and serum IgE are described in WO 2021/195530, incorporated by reference herein.

### Results

A total of 162 patients were randomized (79 to placebo+TCS; 83 to dupilumab+TCS). Of these patients, only one was not treated (placebo group). Of the patients who were treated, almost all completed Week 16 (94.9% for placebo+TCS; 98.8% for dupilumab+TCS; 96.9% of total patients).

Baseline demographics and disease characteristics are summarized in Tables 1 and 2. Baseline demographics were generally balanced between the treatment arms; relatively few patients were in the lower age subgroup (6 months to <2 years) but a sizeable number where in the lower weight group (5 to <15 kg). Baseline disease characteristics were balanced across the treatment arms. The study population had a high baseline disease severity as reflected by measures of signs, symptoms, and quality of life. Additionally, a high percentage of patients had prior use of systemic medications for AD (28.2% for placebo+TCS group; 28.9% for dupilumab+TCS group; 28.6% of total patients), which is suggestive of patients with severe disease. There was prior systemic corticosteroid use for 17.9% of placebo+TCS patients and 19.3% of dupilumab+TCS patients (18.6% overall). There was prior systemic non-steroidal immunosuppressant use (azathioprine, cyclosporine, methotrexate, or mycophenolate) by 15.4% of placebo+TCS patients and 15.7% of dupilumab+TCS patients (15.5% overall).

TABLE 1
Baseline Demographics

| | Placebo + | | Dupilumab + | | | |
|---|---|---|---|---|---|---|
| | TCS | | TCS | | Overall | |
| n (Full Analysis Set (FAS)) | 79 | | 83 | | 162 | |
| Age (years), mean (SD) | 3.78 | (1.262) | 3.91 | (1.225) | 3.8 | (1.24) |
| ≥6 mo to < 2 yr | 5 | (6.3%) | 6 | (7.2%) | 11 | (6.8%) |
| ≥2 yr to < 6 yr | 74 | (93.7%) | 77 | (92.8%) | 151 | (93.2%) |
| Gender (Male), n (%) | 55 | (69.6%) | 44 | (53.0%) | 99 | (61.1%) |
| Race, n (%) | | | | | | |
| White | 53 | (67.1%) | 58 | (69.9%) | 111 | (68.5%) |
| Black or African American | 16 | (20.3%) | 14 | (16.9%) | 30 | (18.5%) |
| Asian | 4 | (5.1%) | 6 | (7.2%) | 10 | (6.2%) |
| Other | 4 | (5.1%) | 3 | (3.6%) | 7 | (4.3%) |
| Weight (kg), mean (SD) | | | | | | |
| 5 to < 15 kg | 25 | (31.6%) | 26 | (31.3%) | 51 | (31.5%) |
| 15 to < 30 kg | 54 | (68.4%) | 57 | (68.7%) | 111 | (68.5%) |

TABLE 2
Baseline Disease Characteristics

| | Placebo + TCS | | Dupilumab + TCS | | Overall | |
|---|---|---|---|---|---|---|
| n (FAS) | 79 | | 83 | | 162 | |
| Duration of AD (yr), mean (SD) | 3.4 | (1.30) | 3.4 | (1.33) | 3.4 | (1.31) |
| Age at onset < 6 months, n (%) | 57 | (72.2) | 50 | (60.2) | 107 | (66.0) |
| Age at onset ≥ 6 months, n (%) | 22 | (27.8) | 33 | (39.8) | 55 | (34.0) |
| EASI (0-72), mean (SD) | 33.1 | (12.18) | 35.1 | (13.88) | 34.1 | (13.08) |
| IGA (0-4), n (%) | | | | | | |
| 3 | 17 | (21.5%) | 20 | (24.1%) | 37 | (22.8%) |
| 4 | 62 | (78.5%) | 63 | (75.9%) | 125 | (77.2%) |
| Peak Pruritus NRS (0-10), mean (SD) | 7.6 | (1.49) | 7.5 | (1.32) | 7.6 | (1.40) |
| BSA involvement (%), mean (SD) | 57.4 | (20.91) | 59.3 | (22.51) | 58.4 | (21.70) |
| POEM (0-28), mean (SD) | 23.3 | (4.0) | 23.1 | (4.5) | 23.2 | (4.3) |
| SCORAD (0-103), mean (SD) | 72.2 | (11.44) | 72.7 | (12.95) | 72.4 | (12.20) |
| cDLQI* (0-30), mean (SD) | 17.7 | (6.25) | 17.5 | (5.43) | 17.6 | (5.77) |
| | (n = 38) | | (n = 48) | | | |
| IDQoL* (0-30), mean (SD) | 17.1 | (5.37) | 17.4 | (5.41) | 17.2 | (5.35) |
| | (n = 41) | | (n = 35) | | | |
| DFI* (0-30), mean (SD) | 17.6 | (7.24) | 17.2 | (5.99) | 17.4 | (6.61) |
| Peak skin pain NRS (0-10), mean (SD) | 7.2 | (1.84) | 6.8 | (1.76) | 7.0 | (1.81) |
| Patient sleep quality NRS (0-10), mean | 4.6 | (2.09) | 4.9 | (1.90) | 4.8 | (2.00) |
| (SD) | | | | | | |
| Prior use of systemic medications for | 22 | (28.2) | 24 | (28.9) | 46 | (28.6) |
| AD, n (%) | | | | | | |
| Prior use of systemic corticosteroids, n | 14 | (17.9) | 16 | (19.3) | 30 | (18.6) |
| (%) | | | | | | |
| Prior use of systemic non-steroidal | 12 | (15.4) | 13 | (15.7) | 25 | (15.5) |

immunosuppressants, n (%)

*cDLQI: Child dermatology life quality index; assessed in pediatric patients ≥4-<18 yrs.
IDQoL: Infant dermatitis quality of life; assessed in patients <4 yrs.
DFI: Dermatitis family index; assessed in caregivers

As shown in Table 3, there was a high incidence of atopic co-morbidities in the patient population, underscoring the common Type 2 pathophysiology behind these diseases.

TABLE 3
Concurrent Atopic/Allergic Conditions

| | Placebo + | | Dupilumab + | | | |
|---|---|---|---|---|---|---|
| | TCS | | TCS | | Overall | |
| n (FAS) | 78 | | 83 | | 161 | |
| # of patients with ≥1 | 65 | (83.3%) | 66 | (79.5%) | 131 | (81.4%) |
| concurrent | | | | | | |
| allergic condition | | | | | | |
| Allergic Rhinitis | 36 | (46.2%) | 35 | (42.2%) | 71 | (44.1%) |
| Asthma | 21 | (26.9%) | 20 | (24.1%) | 41 | (25.5%) |
| Food Allergy | 55 | (70.5%) | 55 | (66.3%) | 110 | (68.3%) |
| Allergic Conjunctivitis | 3 | (3.8%) | 4 | (4.8%) | 7 | (4.3%) |
| Hives | 15 | (19.2%) | 14 | (16.9%) | 29 | (18.0%) |
| Chronic Rhinosinusitis | 2 | (2.6%) | 1 | (1.2%) | 3 | (1.9%) |
| Nasal Polyps | 0 | | 0 | | 0 | |
| Eosinophilic Esophagitis | 1 | (1.3%) | 2 | (2.4%) | 3 | (1.9%) |
| Other Allergies* | 42 | (53.8%) | 43 | (51.8%) | 85 | (52.8%) |

*Refers to allergies to plants, animals, dust mite, medication, etc.

### Efficacy

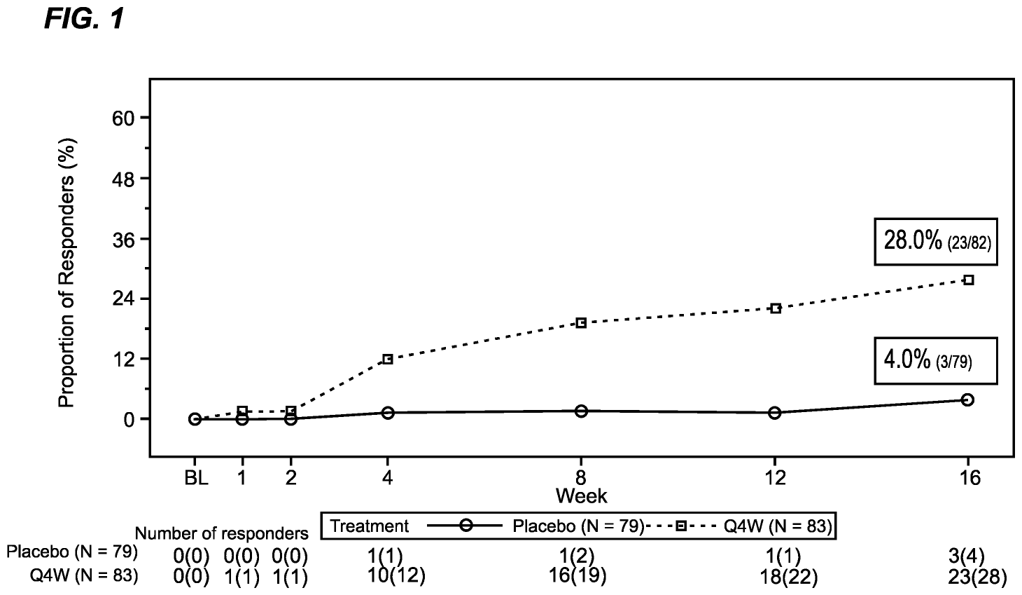

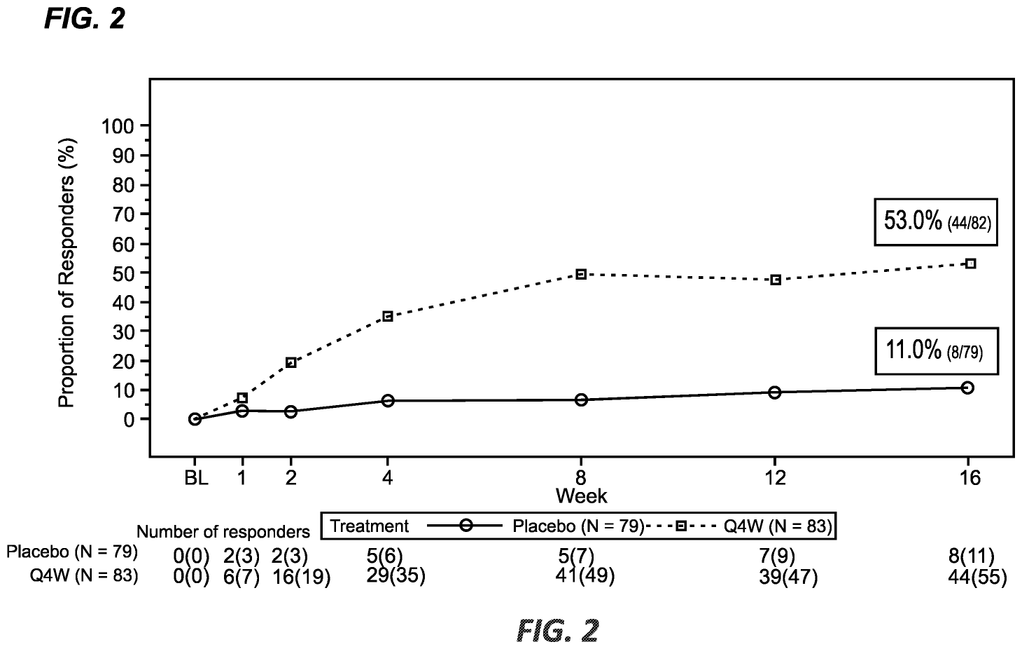

Treatment with dupilumab+TCS significantly improved all prespecified efficacy endpoints. The primary endpoints assessed the proportion of patients achieving an Investigator's Global Assessment (IGA) score of 0 (clear) or 1 (almost clear) and 75% improvement in Eczema Area and Severity Index (EASI-75). At 16 weeks, 28% of patients treated with dupilumab achieved clear or almost-clear skin compared to 4% with placebo (p<0.0001); a statistically significant difference between the dupilumab and placebo arms was apparent starting at Week 4 and continuing through Week 16 (FIG. 1). 53% of patients treated with dupilumab achieved 75% or greater skin improvement from baseline compared to 11% with placebo (p<0.0001); a statistically significant difference between the dupilumab and placebo arms was apparent starting at Week 2 and continuing through Week 16 (FIG. 2).

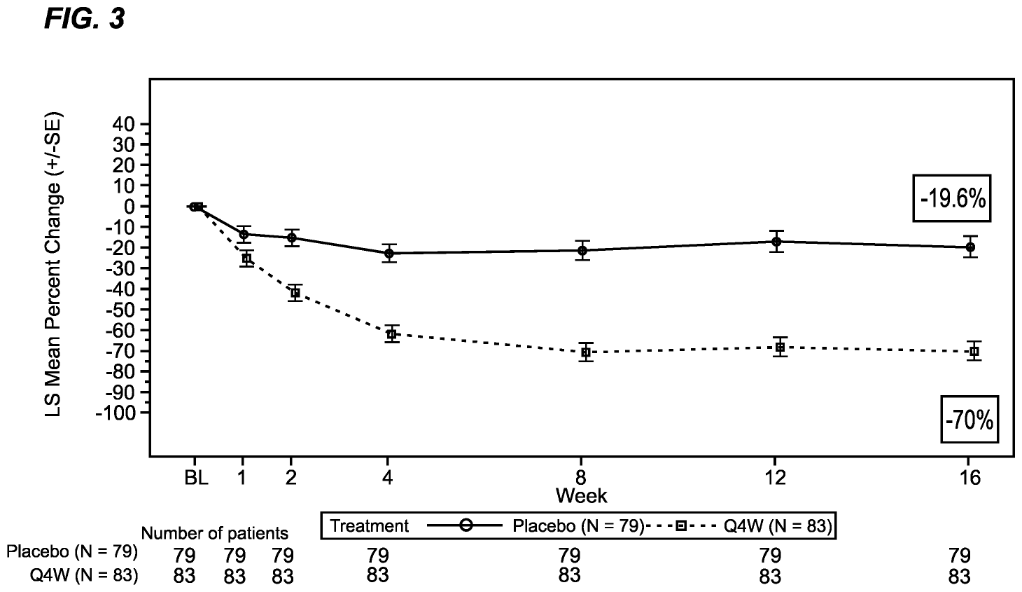

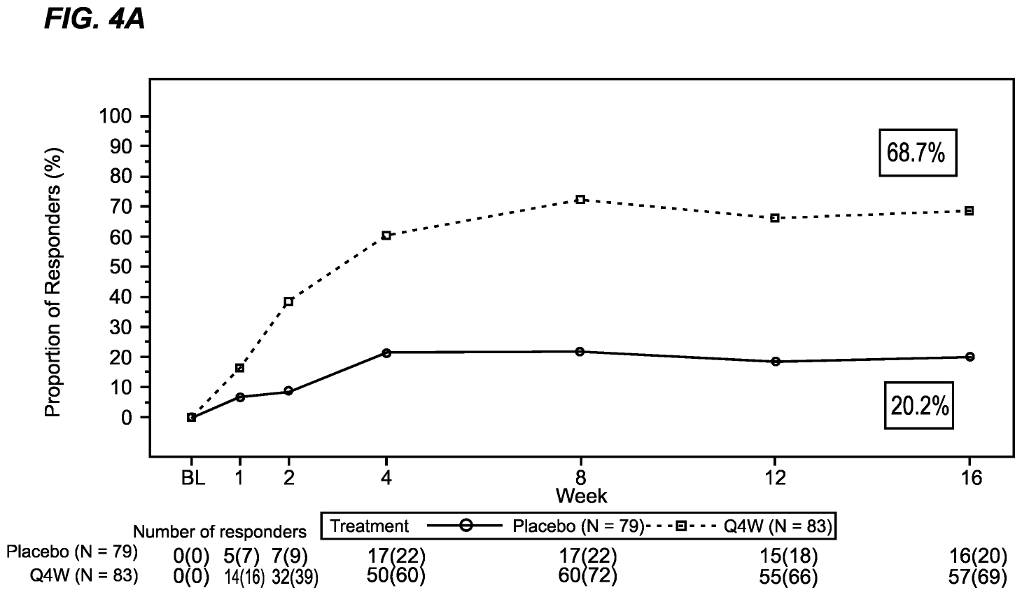

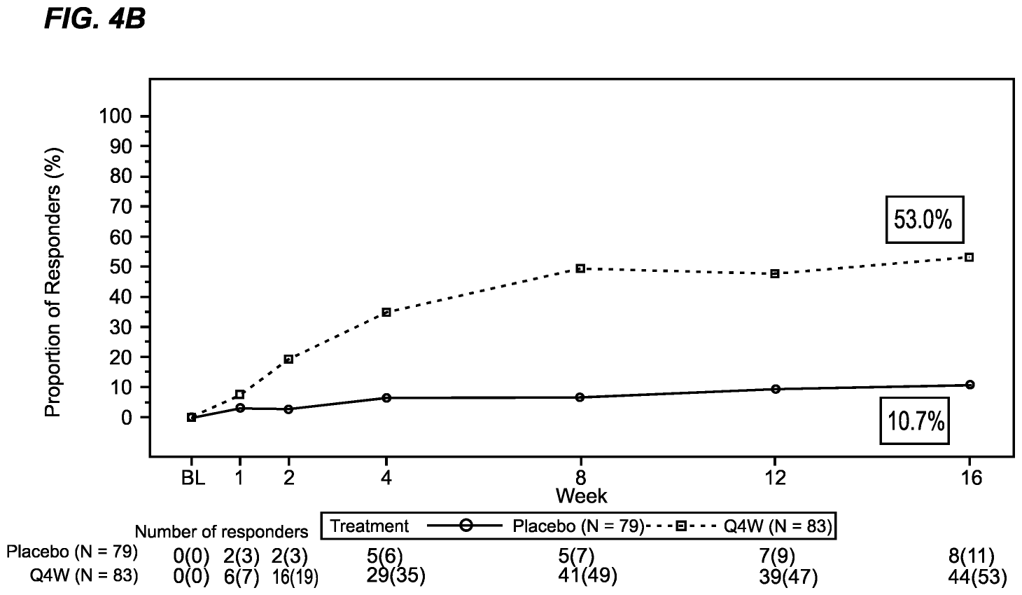

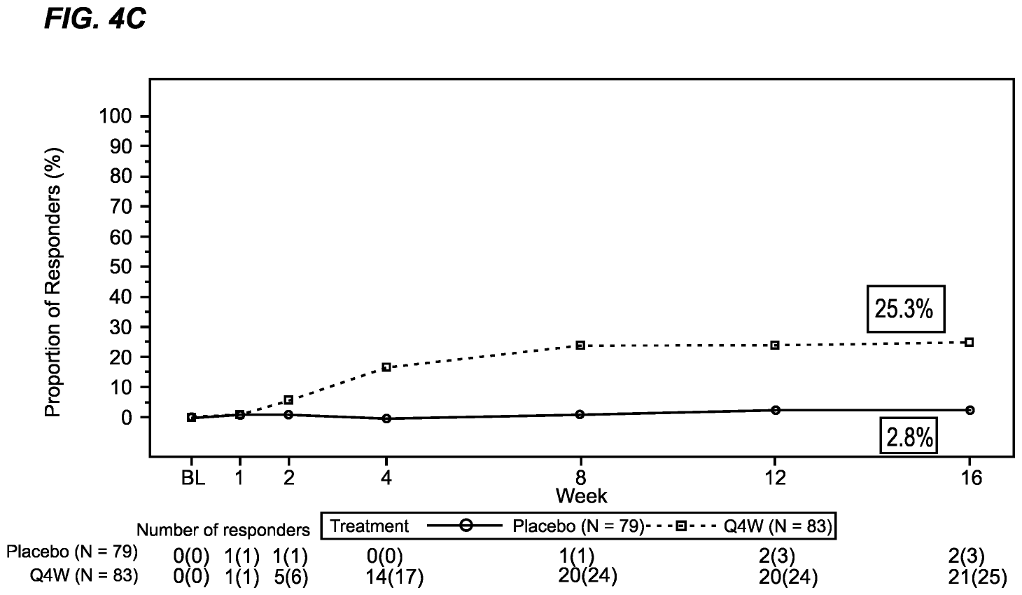

Treatment with dupilumab resulted in rapid and sustained improvements in the extent and severity of physical signs of AD, as measured by EASI. On average, dupilumab-treated patients had a 70% average improvement from baseline in EASI score at Week 16, compared to 20% improvement with placebo (p<0.0001). Statistically significant improvements were seen as early as Week 1 and were sustained through Week 16 (FIG. 3). A significantly greater proportion of dupilumab-treated patients achieved EASI-50 (FIG. 4A), EASI-75 (FIG. 4B), and EASI-90 (FIG. 4C) at Week 16, with many patients achieving EASI-50 as early as Week 1, achieving EASI-75 as early as Week 2, and achieving EASI-90 as early as Week 4.

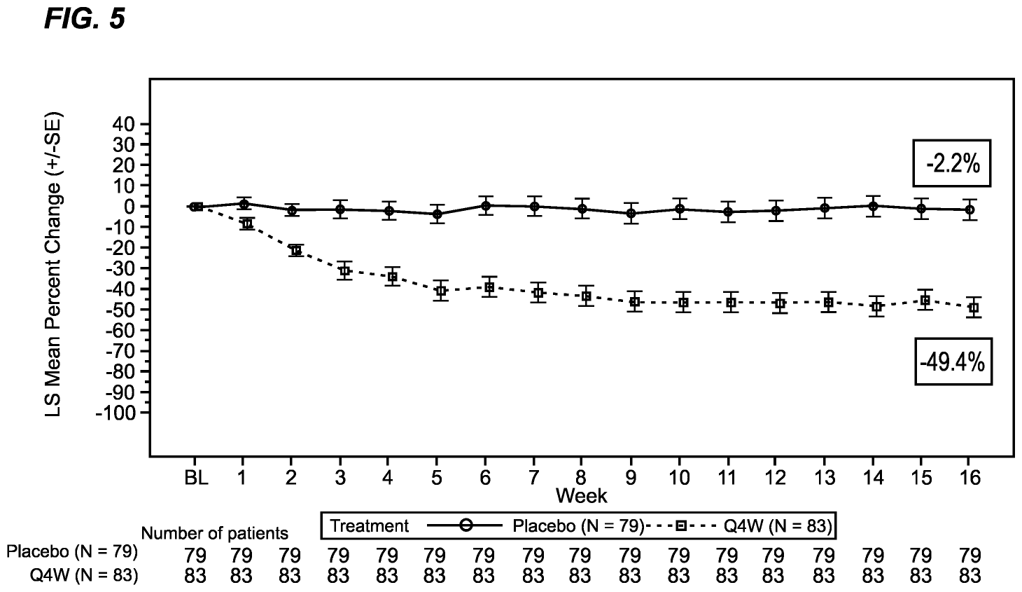

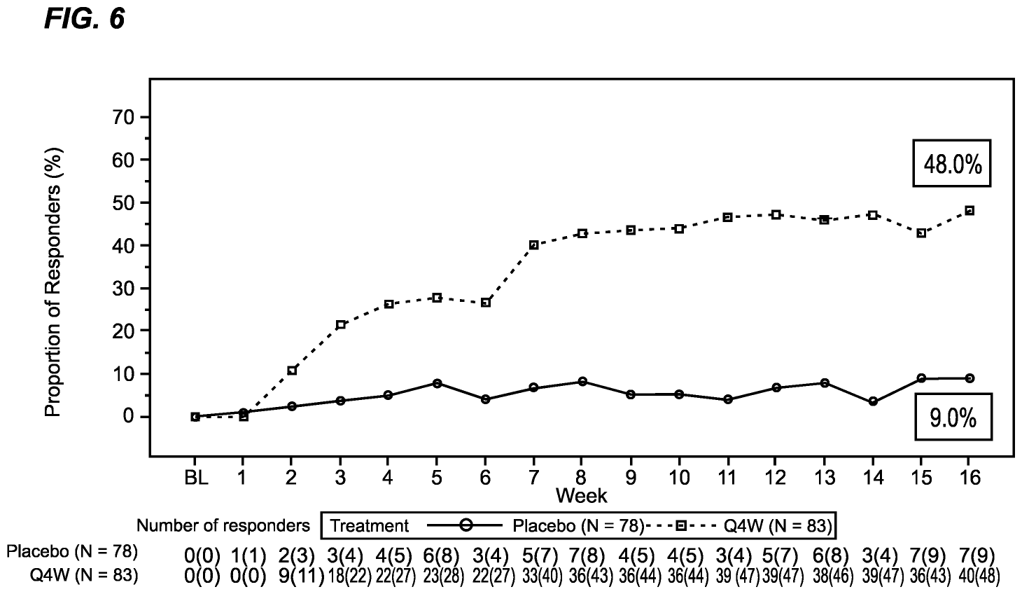

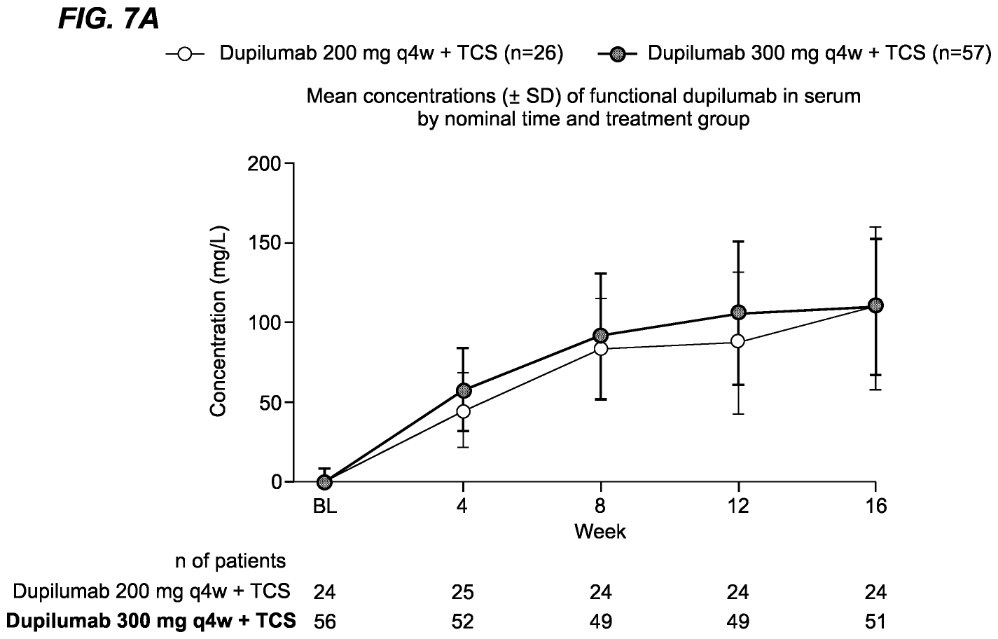

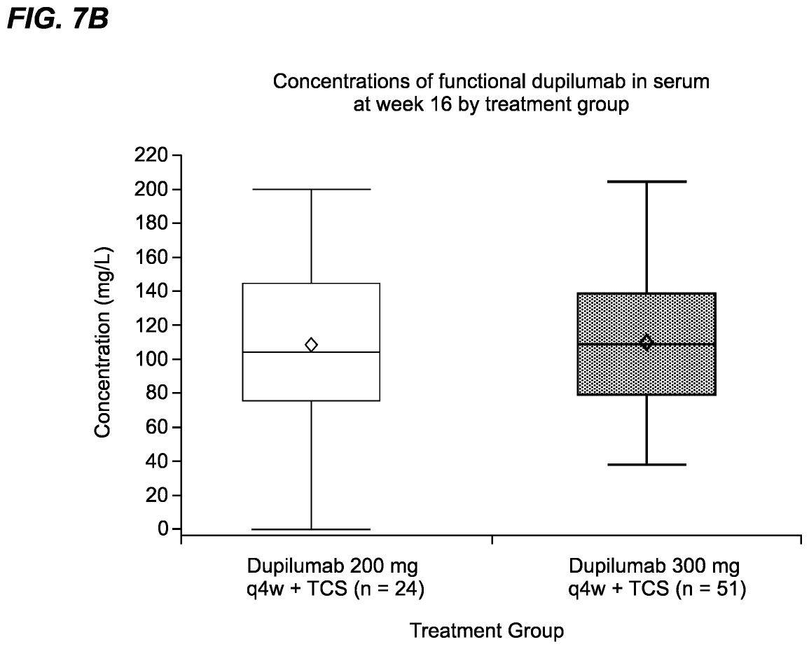

Treatment with dupilumab rapidly improved pruritus symptoms (as early as Week 1), and these improvements in pruritus were sustained through Week 16. See FIG. 5. At Week 16 dupilumab-treated patients showed a 49% average improvement from baseline in itch compared to 2% improvement with placebo (p<0.0001). From Week 3 onward, a significantly greater proportion of dupilumab-treated patients achieved 4-point improvements in pruritus NRS from baseline as compared to the placebo arm (FIG. 6).

Improvements in AD signs across anatomical regions was assessed using unweighted EASI body region scores (range 0-72). Baseline mean (SE) imputed EASI body region scores in the dupilumab/placebo groups were: head, 28.8 (18.1)/24.5 (15.5); trunk, 29.7 (16.3)/27.8 (15.1); upper extremities, 40.3 (15.7)/38.2 (14.7); and lower extremities, 41.3 (17.3)/40.6 (15.4). At Week 16, the LS mean (SE) unweighted EASI body regions scores in the dupilumab/placebo groups were: head, 11.0 (1.9)/25.3 (1.9); trunk, 9.5 (1.8)/25.2 (1.9); upper extremities, 13.9 (2.2)/33.9 (2.3); and lower extremities, 14.6 (2.2)/35.3 (2.3); P<0.0001 for dupilumab vs placebo, all regions. Improvement in all regions was seen as early as Week 2 (P<0.0001 for dupilumab vs placebo).

Improvements in individual EASI components (e.g., excoriations, erythema, infiltration/papulation, and lichenification) were also observed. Improvement in excoriations signs across anatomical regions was assessed using excoriations EASI sign scores (0-3). Baseline mean (SE) imputed excoriations EASI sign scores in the dupilumab/placebo groups were: head, 1.7 (1.0)/1.5 (1.0); trunk, 1.8 (0.9)/1.7 (1.0); upper extremities, 2.5 (0.7)/2.4 (0.8); and lower extremities, 2.3 (0.8)/2.4 (0.8). At Week 16, the LS mean (SE) excoriations EASI sign scores in the dupilumab/placebo groups were: head, 0.7 (0.1)/1.6 (0.1); trunk, 0.7 (0.1)/1.6 (0.1); upper extremities, 0.9 (0.1)/2.0 (0.1); and lower extremities, 1.0 (0.1)/2.0 (0.1); P<0.0001 for dupilumab vs placebo, all regions. Improvement in all regions was seen as early as Week 2 (P<0.001 for dupilumab vs placebo).

Improvement in erythema signs across anatomical regions was assessed using erythema EASI sign scores (0-3). Baseline mean (SE) imputed erythema EASI sign scores in the dupilumab/placebo groups were: head, 2.1 (0.8)/2.0 (0.8); trunk, 2.1 (0.8)/2.1 (0.7); upper extremities, 2.4 (0.6)/2.5 (0.5); and lower extremities, 2.5 (0.6)/2.6 (0.5). At Week 16, the LS mean (SE) erythema EASI sign scores in the dupilumab/placebo groups were: head, 1.3 (0.1)/2.0 (0.1); trunk, 1.0 (0.1)/1.7 (0.1); upper extremities, 1.2 (0.1)/2.1 (0.1); and lower extremities, 1.3 (0.1)/2.2 (0.1); P<0.0001 for dupilumab vs placebo, all regions. Improvement in all regions was seen as early as Week 2 (P<0.01 for dupilumab vs placebo).

Improvement in infiltration/papulation signs across anatomical regions was assessed using infiltration/papulation EASI sign scores (0-3). Baseline mean (SE) imputed infiltration/papulation EASI sign scores in the dupilumab/placebo groups were: head, 1.7 (0.9)/1.6 (0.9); trunk, 2.0 (0.8)/1.8 (0.8); upper extremities, 2.3 (0.6)/2.3 (0.6); and lower extremities, 2.3 (0.7)/2.4 (0.6). At Week 16, the LS mean (SE) infiltration/papulation sign scores in the dupilumab/placebo groups were: head, 0.9 (0.1)/1.6 (0.1); trunk, 0.8 (0.1)/1.6 (0.1); upper extremities, 1.0 (0.1)/1.9 (0.1); and lower extremities, 1.1 (0.1)/2.0 (0.1); P<0.0001 for dupilumab vs placebo, all regions.

Improvement in lichenification signs across anatomical regions was assessed using lichenification EASI sign scores (0-3). Baseline mean (SE) imputed lichenification EASI sign scores in the dupilumab/placebo groups were: head, 1.7 (1.0)/1.5 (1.0); trunk, 1.6 (0.9)/1.6 (0.8); upper extremities, 2.3 (0.6)/2.3 (0.7); and lower extremities, 2.4 (0.6)/2.4 (0.7). At Week 16, the LS mean (SE) lichenification sign scores in the dupilumab/placebo groups were: head, 0.9 (0.1)/1.5 (0.1); trunk, 0.8 (0.1)/1.3 (0.1); upper extremities, 1.3 (0.1)/1.9 (0.1); and lower extremities, 1.2 (0.1)/2.0 (0.1); P<0.0001 for dupilumab vs placebo, all regions.

Improvement in EASI area score was assessed for each of the head, trunk, upper extremities, and lower extremities regions. Baseline mean (SE) imputed EASI area scores (0-6 scale) in the dupilumab/placebo groups were: head, 3.7 (1.6)/3.4 (1.5); trunk, 3.8 (1.5)/3.7 (1.3); upper extremities, 4.2 (1.3)/4.0 (1.3); and lower extremities, 4.3 (1.3)/4.1 (1.3). At Week 16, the LS mean (SE) EASI area scores in the dupilumab/placebo groups were: head, 2.0 (0.2)/3.2 (0.2); trunk, 1.5 (0.2)/3.1 (0.2); upper extremities, 2.0 (0.2)/3.5 (0.2); and lower extremities, 2.0 (0.2)/3.6 (0.2); P<0.0001 for dupilumab vs placebo, all regions.

Significant improvements in measures of observed patient outcomes (including sleep, skin pain, and health-related quality of life), as well as caregiver-reported health-related quality of life, were also observed for patients treated with dupilumab. Tables 4 and 5 summarize improvements in various AD-associated parameters in patients treated with dupilumab+low potency TCS, as compared to placebo+low potency TCS, for the overall population (Table 4) and in weight subgroup analyses (Table 5). As shown in Table 4, treatment with dupilumab resulted in a LS mean change in skin pain NRS of −3.9 at Week 16, versus −0.62 for placebo-treated patients. The reduction in skin pain NRS was rapid (as early as Week 1) and sustained through Week 16. Additionally, a significant proportion of patients exhibited a ≥4-point improvement in skin pain NRS (47.2% at Week 16).

In a weight subgroup analysis (5 to <15 kg and 15 to <30 kg), dupilumab showed superiority over placebo consistently across both weight groups (see Table 5). Both weight subgroups showed rapid onset of effect of treatment with dupilumab, for example as measured by mean percent change in EASI over time and mean percent change in pruritus NRS score over time. Additionally, there was a trend for a numerically higher effect dupilumab versus placebo in patients <2 years old (Table 6).

TABLE 4
Efficacy Results for Co-Primary and Secondary Endpoints

| | | | | | | | | P-value |
|---|---|---|---|---|---|---|---|---|
| | | Placebo | | Dupilumab | | Change vs. placebo | | vs. |
| Level | Week 16 Endpoints | (n = 79) | | (n = 83) | | (95% Cl) | | placebo |
| Primary/ | Proportion of patients with IGA 0 to 1 (score range | 3 | (3.9) | 23 | (27.7) | 23.8 | (13.27, 34.37) | <0.0001 |
| Co-Primary | 0-4), n (%) | | | | | | | |
| | Proportion of patients with EASI-75 (score range 0- | 8 | (10.7) | 44 | (53.0) | 42.3 | (29.47, 55.16) | <0.0001 |
| | 72), n (%) | | | | | | | |
| Key | Mean percent change from baseline in EASI, LS | −19.6 | (5.13) | −70.0 | (4.85) | −50.4 | (−62.38, −38.40) | <0.0001 |
| Secondary | Mean (SE) | | | | | | | |
| | Mean percent change from baseline in Pruritus NRS | −2.2 | (5.22) | −49.4 | (5.03) | −47.2 | (−59.47, −34.79) | <0.0001 |
| | (score range 0-10), LS Mean (SE) | | | | | | | |
| Other | Proportion of patients with improvement of pruritus | 7 | (8.9) | 40 | (48.1) | 39.2 | (26.18, 52.27) | <0.0001 |
| Secondary | NRS (score range 0-10) ≥4, n (%) | | | | | | | |
| | Proportion of patients with improvement of pruritus | 8 | (9.9) | 44 | (53.3) | 43.4 | (30.03, 56.67) | <0.0001 |
| | NRS (score range 0-10) ≥3, n (%) | | | | | | | |
| | Proportion of patients with EASI-50, n (%) | 16 | (20.2) | 57 | (68.7) | 48.5 | (35.03, 62.00) | <0.0001 |
| | Proportion of patients with EASI-90, n (%) | 2 | (2.8) | 21 | (25.3) | 22.5 | (12.37, 32.60) | <0.0001 |
| | Mean change from baseline in percent BSA affected | −10.74 | (2.926) | −35.00 | (2.815) | −24.3 | (−31.204, −17.329) | <0.0001 |
| | by AD, LS Mean (SE) | | | | | | | |
| | Mean change from baseline in POEM (scale range | −3.8 | (0.92) | −12.9 | (0.89) | −9.1 | (−11.26, −6.89) | <0.0001 |
| | 0-28), LS Mean (SE) | | | | | | | |
| | Mean percent change from baseline in SCORAD | −16.2 | (3.54) | −54.7 | (3.39) | −38.4 | (−46.65, −30.21) | <0.0001 |
| | (score range 0-103), LS Mean (SE) | | | | | | | |
| | Mean change from baseline in patient's sleep quality | 0.34 | (0.256) | 2.04 | (0.251) | 1.7 | (1.092, 2.317) | <0.0001 |
| | NRS* (0-10), LS Mean (SE) | | | | | | | |
| | Mean change from baseline in patient's skin pain | −0.62 | (0.302) | −3.93 | (0.295) | −3.31 | (−4.029, −2.600) | <0.0001 |
| | NRS (range 0-10), LS Mean (SE) | | | | | | | |
| | Mean change from baseline in DFI (score range 0- | −2.68 | (0.839) | −10.48 | (0.806) | −7.8 | (−9.789, −5.814) | <0.0001 |
| | 30), LS Mean (SE) | | | | | | | |
| | Mean change from baseline in CDLQI (range 0-30), | −2.5 | (1.66) | −10.0 | (1.56) | −7.5 | (−10.29, −4.75) | <0.0001 |
| | LS Mean (SE) (n = 47 in dupilumab, n = 38 in placebo) | | | | | | | |
| | Mean change from baseline in IDQOL (range 0-30), | −1.95 | (1.078) | −10.91 | (1.159) | −8.96 | (−11.711, −6.202) | <0.0001 |

LS Mean (SE) (n = 36 in dupilumab, n = 41 in placebo)

*Increase in score means improvement

TABLE 5
Overall Efficacy by Dose and Weight Group

| | ≥5 kg to <15 kg | | | ≥15 kg to <30 kg | | |
|---|---|---|---|---|---|---|
| | | DPL | Difference | | DPL | Difference |
| | | 200 mg | vs. | | 300 mg | vs. |
| | Placebo | Q4W | placebo | Placebo | Q4W | placebo |
| Endpoint at Week 16 | n = 25 | n = 26 | (95% CI) | n = 54 | n = 57 | (95% CI) |
| IGA 0-1 at week 16, n (%) | 1 | 10 | 34.4 | 2 | 13 | 19.0 |
| | (4.1) | (38.5) | (14.07, 54.65) | (3.8) | (22.8) | (6.96, 31.06) |
| EASI-75 at week 16, n (%) | 2 | 15 | 48.7 | 6 | 29 | 39.4 |
| | (9.0) | (57.7) | (26.37, 71.01) | (11.5) | (50.9) | (23.81, 54.98) |
| % change EASI score from baseline to week | −14.6 | −57.3 | −42.7 | −10.1 | −65.5 | −55.4 |
| 16, LS Mean (SE) | (8.77) | (8.20) | (−66.24, −19.21) | (5.29) | (5.13) | (−69.86, −40.89) |
| % change Peak Pruritus NRS from baseline to | 11.5 | −44.0 | −55.5 | −5.6 | −47.3 | −41.7 |
| week 16, LS Mean (SE) | (10.60) | (10.27) | (−84.40, −26.54) | (3.98) | (3.89) | (−52.62, −30.78) |
| ≥4 point reduction in Peak Pruritus NRS from | 1 | 13 | 46.0 | 6 | 27 | 36.2 |
| baseline to week 16, n (%) | (5.5) | (51.5) | (24.13, 67.90) | (10.4) | (46.5) | (20.26, 52.07) |
| ≥3 point reduction in Peak Pruritus NRS from | 2 | 15 | 49.3 | 6 | 30 | 40.7 |
| baseline to week 16, n (%) | (6.8) | (56.1) | (26.70, 71.87) | (11.3) | (52.0) | (24.55, 56.80) |
| Proportion of patients with EASI-50 at week | 4 | 16 | 44.0 | 12 | 41 | 50.5 |
| 16, n (%) | (17.5) | (61.5) | (19.83, 68.25) | (21.4) | (71.9) | (34.45, 66.64) |
| Proportion of patients with EASI-90 at week | 0 | 9 | 33.8 | 2 | 12 | 17.3 |
| 16, n (%) | | (34.6) | (14.93, 52.70) | (3.8) | (21.1) | (5.55, 29.05) |
| Change in % BSA affected by AD from | −8.1 | −29.5 | −21.4 | −7.8 | −33.2 | −25.9 |
| baseline to week 16, LS Mean (SE) | (5.19) | (5.05) | (−35.60, −7.21) | (2.96) | (2.88) | (−34.05, −17.84) |
| Change in POEM from baseline to week 16, | −1.9 | −11.3 | −9.4 | −2.8 | −11.8 | −9.1 |
| LS Mean (SE) | (1.58) | (1.52) | (−13.70, −5.12) | (0.98) | (0.95) | (−11.73, −6.39) |
| % change in SCORAD from baseline to week | −13.9 | −46.4 | −32.5 | −10.3 | −51.5 | −41.2 |
| 16, LS Mean (SE) | (5.58) | (5.32) | (−47.66, −17.40) | (3.59) | (3.48) | (−50.98, −31.39) |
| Change in patient's sleep quality NRS from | 0.1 | 1.5 | 1.4 | 0.3 | 2.1 | 1.8 |
| baseline to week 16, LS Mean (SE) | (0.36) | (0.35) | (0.44, 2.43) | (0.28) | (0.28) | (1.05, 2.59) |
| Change in patient's skin pain NRS from | 0 | −3.5 | −3.5 | −0.6 | −3.8 | −3.3 |
| baseline to week 16, LS Mean (SE) | (0.50) | (0.47) | (−4.85, −2.18) | (0.31) | (0.31) | (−4.11, −2.38) |

TABLE 6
Efficacy Outcomes in Patients < 2 Years of Age

| | | | Dupilumab 200/300 | |
|---|---|---|---|---|
| | Placebo | | mg Q4W | |
| Efficacy Outcomes | (n = 5) | | (n = 6) | |
| IGA 0/1 at week 16, n (%) | 1 | (20) | 2 | (33) |
| EASI-75 at week 16, n (%) | 1 | (25) | 4 | (67) |
| % change in EASI from baseline to | −30.0 | (35.6) | −64.6 | (42.1) |
| week 16, LS mean (SE) | | | | |
| % change in worst scratch/itch | −5.5 | (11.8) | −52.4 | (39.8) |

NRS from baseline to week 16, LS
mean (SE)

### Use of Rescue Medication

A high proportion of patients in the placebo arm needed rescue with at least one rescue medication (49 of 79 patients; 62.0%). In contrast, the use of rescue was substantially lower in the dupilumab treatment arm, with separation from placebo evident as early as Week 2 (16 of 83 patients; 19.3%). The most commonly used dermatological rescue medication by therapeutic class was dermatological preparations of corticosteroids. Systemic corticosteroids were used for rescue of exacerbation of AD in 2 ( 2/78; 3%) patients in the placebo group and 1 ( 1/83; 1%) patients in the dupilumab group. Mean weekly dose of medium-to-high potency TCS rescue was lower with dupilumab than placebo (7.8 g vs. 9.2 g, respectively).

### Safety

Dupilumab was well tolerated and demonstrated an acceptable safety profile, with no new safety concerns identified. See Table 7. For the 16-week treatment period, overall rates of adverse events (AEs) were 64% for dupilumab and 74% for placebo. Notably, 50% fewer patients treated with dupilumab experienced skin infection as compared to the placebo arm. Most common AEs and AEs of special interest included skin infections (12% dupilumab, 24% placebo), nasopharyngitis (8% dupilumab, 9% placebo), upper respiratory tract infection (6% dupilumab, 8% placebo), conjunctivitis (5% dupilumab, 0% placebo), herpes viral infections (6% dupilumab, 5% placebo) and injection site reactions (2% dupilumab, 3% placebo). The percentage of patients with 1 skin infections was numerically lower in the dupilumab group (12.0%) than in the placebo group (24.4%).

When exposure-adjusted rates (patients with 1 event per 100 patient-years [nP/100PY] were calculated, total infection rates were found to be numerically lower in the dupilumab-treated group (nP/100PY: 185.2) compared with the placebo-treated group (nP/100PY: 245.7). Infection rates in skin structures and soft tissues were also numerically lower in dupilumab-treated patients (nP/100PY: 24.7) than in placebo-treated patients (nP/100PY: 40.2). Bacterial infections were significantly less frequent in dupilumab-treated (nP/100PY: 3.9) than placebo-treated patients (nP/100PY:45.6; P<0.05 vs placebo). Additionally, non-herpetic skin infections were significantly lower in the dupilumab group (nP/100PY: 42.7) compared with placebo (nP/100PY: 92.7; P<0.05 vs placebo). Significantly lower rates of systemic anti-infective medication use were reported with dupilumab (nP/100PY: 104.7) compared with placebo (nP/100PY: 203.0; P<0.05 vs placebo).

Rates of fungal infections were not significantly different with dupilumab (nP/100PY: 0) than with placebo (nP/100PY: 4.2; P=1.0 vs placebo). There was no significant difference between the dupilumab and placebo in the rates of viral infections (dupilumab nP/100PY: 64.8; placebo nP/100PY: 55.2; P=0.681 vs placebo) or herpetic infections (dupilumab nP/100PY: 20.0; placebo nP/100PY: 17.1; P=0.817 vs placebo). Molluscum contagiosum rates were numerically higher in the dupilumab group (nP/100PY: 15.9) than in the placebo group (nP/100PY: 8.4). No helminthic infections were reported in either group.

Hematology and chemistry laboratory safety data was evaluated. At baseline, mean (SD) counts of hematology parameters were similar in both treatment groups: hemoglobin (dupilumab: 129.4 g/L [12]; placebo: 127.2 g/L [11.4]), lymphocyte (dupilumab: 4.6×10⁹/L [1.8]; placebo: 4.5×10⁹/L [1.7]), basophil (dupilumab: 0.07×10⁹/L [0.03]; placebo: 0.07×10⁹/L [0.04]), platelet (dupilumab: 397.7×10⁹/L [103.2]; placebo: 385.6×10⁹/L [112.9]), and eosinophils (dupilumab: 1.1×10⁹/L [0.7]; placebo: 1.1×10⁹/L [0.7]). Mean (SD) hemoglobin count in the dupilumab (128.4×g/L[11]) and placebo groups (128.2×g/L [11.2]), lymphocyte count in the dupilumab (4.20×10⁹/L [2.06]) and placebo groups (4.29×10⁹/L [1.52]) and basophil count in the dupilumab (0.07×10⁹/L [0.04]) and placebo groups (0.06×10⁹/L [0.03]) remained with the normal reference range for this population at Week 16. Mean change (SD) in platelet count at Week 16 was −16.3×10⁹/L (78.5) in the dupilumab group and +17.4×10⁹/L (106.6) in the placebo group. In the dupilumab treatment group the mean eosinophil count increased at Week 4 (mean change from baseline [SD]; +0.48×10⁹/L [1.8]) and trended downward by Week 16 (+0.31×10⁹/L [1.4]) while minimal changes were noted in the placebo group at Week 4 (0.1×10⁹ [0.7]) and Week 16 (−0.2×10⁹ [0.7]). The values for creatine kinase, alkaline phosphatase, lactate dehydrogenase, blood urea nitrogen, albumin and protein at Week 16 remained within the normal reference range in all treatment groups. Two patients in the dupilumab 200/300 mg Q4W arm of this study reported treatment emergent adverse events of severe and moderate eosinophilia. Neither event was associated with clinical symptoms or led to discontinuation of study treatment.

TABLE 7
Treatment-Emergent Adverse Events

| | Placebo + TCS | | Dupilumab + TCS | |
|---|---|---|---|---|
| % Patients | n = 78 | | n = 83 | |
| Deaths | 0 | | 0 | |
| TEAEs | 58 | (74.4%) | 53 | (63.5%) |
| SAEs | 4 | (5.1%) | 0 | |
| AEs leading to discontinuation | 1 | (1.3%) | 1 | (1.2%) |
| TEAE of Special Interest | 0 | | 1 | (1.2%) |
| Conjunctivitis (Narrow CMQ) | 0 | | 4 | (4.8%) |
| Conjunctivitis allergic | 0 | | 1 | (1.2%) |
| Conjunctivitis | 0 | | 3 | (3.6%) |
| Conjunctivitis (Broad CMQ) | 1 | (1.3%) | 6 | (7.2%) |
| Skin infection | 19 | (24.4%) | 10 | (12%) |
| Injection site reactions (HLT) | 2 | (2.6%) | 2 | (2.4%) |
| Herpes viral infections (HLT) | 4 | (5.1%) | 5 | (6.0%) |

### Biomarker Analysis

Serum for biomarker analysis was collected from patients at baseline, Week 4, and Week 16. Baseline median serum TARC and total IgE levels for dupilumab/placebo groups (n=83/79) were 3,295/3,190 pg/mL and 2,190/3,240 kU/L, respectively. A significant reduction in TARC from baseline with dupilumab versus placebo was observed as early as Week 4 and maintained through Week 16; after 16 weeks of treatment, the median percentage change from baseline in dupilumab/placebo was −83.1%/−12.8% for TARC (P<0.0001). Serum total IgE decreased from baseline with dupilumab but increased with placebo (−71.2% vs. 28.1%, respectively; P<0.0001). Similar reductions were observed in dupilumab- vs placebo-treated patients for all tested serum allergen-specific IgEs (peanut, egg white, soybean, Dermatophagoides farinae and Dermatophagoides pteronyssinus). The median percent change from baseline in these allergen-specific IgEs at Week 16 was as follows: peanut, −63.9% with dupilumab vs −22.9 with placebo; egg white, −59.8% with dupilumab vs −3.3% with placebo; soybean, −58.0% with dupilumab vs. −14.8% with placebo; Dermatophagoides farinae, −66.2% with dupilumab vs 18.5% with placebo; Dermatophagoides pteronyssinus, −62.9% with dupilumab vs 13.9% with placebo; all P<0.0001). These biomarker results reflect a reduction in systemic type 2 inflammation in dupilumab-treated patients.

### Pharmacokinetics

Mean trough concentrations of functional dupilumab in serum were similar between patients with body weight ≥5 to <15 kg (200 mg dupilumab Q4W) and >15 to <30 kg (300 mg dupilumab Q4W) throughout the treatment period and at week 16 (10⁹ mg/L and 110 mg/L, respectively).

### Conclusion

Dupilumab was evaluated in a Phase 3 trial to treat moderate-to-severe atopic dermatitis in children aged 6 months to ≤6 years. The trial met its primary and all secondary endpoints, showing that adding dupilumab to standard-of-care TCS significantly reduced overall disease severity and improved skin clearance, itch and health-related quality of life at 16 weeks compared to TCS alone. Dupilumab was well tolerated; the trial demonstrated similar safety results to the known safety profile of dupilumab in atopic dermatitis. Additionally, the reduction in serum TARC and total and allergen-specific IgEs in dupilumab-treated patients demonstrates a reduction of systemic type 2 inflammation.

### Example 2: Long-Term Efficacy Data for Dupilumab in Children Aged 6 Months to <6 Years with Moderate-to-Severe Atopic Dermatitis

### Methods

Children aged 6 months to 5 years with moderate-to-severe AD who had participated in the 16-week, double-blind, phase 3 LIBERTY AD PRE-SCHOOL trial (NCT03346434, Part B) were enrolled into an open-label extension (OLE) study (NCT02612454). Patients received subcutaneous dupilumab every 4 weeks (200 mg for children weighing 5 to <15 kg; 300 mg for 15 to <30 kg). Topical AD treatments were allowed.

### Results

Relative to parent study baseline, mean percentage changes (±standard error) in Eczema Area and Severity Index (EASI) score were −41.6 (±4.6) and −54.0 (±3.2) at OLE baseline, −74.5 (±3.7) and −81.7 (±1.8) at Week 16, and −85.6 (±3.5) and −86.4 (±2.2) at Week 52 in the 200 mg and 300 mg dupilumab groups, respectively.

The number of patients (%) achieving an Investigator's Global Assessment (IGA) score of 0/1 increased from OLE baseline ( 6/61 [9.8%] and 15/116 [12.9%]), to Week 16 ( 22/58 [37.9%] and 35/115 [30.4%]), and at Week 52 ( 16/34 [47.1%] and 18/54 [33.3%]) in the 200 mg and 300 mg dupilumab groups, respectively. Overall safety of dupilumab treatment administered for up to 1 year was consistent with the known dupilumab safety profile and no new safety signals emerged.

### Conclusion

Dupilumab treatment for 1 year provides sustained improvement in signs of AD in patients aged 6 months to 5 years with moderate-to-severe AD.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 8
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| | LGS | Dupilumab LCDR2 amino |
| | | acid sequence |
| 8 | MQALQTPYT | Dupilumab LCDR3 amino |
| | | acid sequence |
| 9 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27-6-33-VH |
| | TYYANSVKGRFTISRDNPKNTLFLQMSSLRAEDTAVYYCVRSKVRYRGGMDVWGQ | |
| | GTTVTVSS | |
| 210 | EVQLLESGGGLVQPGGSLRLSCAASGFTLSSYAMHWVRQAPGKGLEYVSGISPSGVS | STSA-C27-7-33-VH |
| | TYYANSVKGRFTISRDNPKNTLFLQMSSLRAEDTAVYYCVRVKVKYRGGMDVWGQ | |
| | GTTVTVSS | |
| 211 | EVQLLESGGGLVQPGGSLRLSCAASGFTLSSYAMHWVRQAPGKGLEYVSGISPTSGS | STSA-C27-24-56-VH |
| | TYYANSVKGRFTISRDNPKNTLFLQMSSLRAEDTAVYYCVRVKVRYRGGMDVWGQ | |
| | GTTVTVSS | |
| 212 | EVQLLESGGGLVQPGGSLRLSCAASGFTLSSYAMHWVRQAPGKGLEYVSGISPTGTS | STSA-C27-47-56-VH |
| | TYYANSVKGRFTISRDNPKNTLFLQMSSLRAEDTAVYYCVRVKGAYRGGMDVWGQ | |
| | GTTVTVSS | |
| 213 | EVQLLESGGGLVQPGGSLRLSCAASGFTLSSYAMHWVRQAPGKGLEYVSGISSSGSS | STSA-C27-33-33-VH |
| | TYYANSVKGRFTISRDNPKNTLFLQMSSLRAEDTAVYYCVRVKVAYRGGMDVWGQ | |
| | GTTVTVSS | |
| 214 | EVQLLESGGGLVQPGGSLRLSCAASGFTLSSYAMHWVRQAPGKGLEYVSGISPSSTS | STSA-C27-56-56-VH |
| | TYYANSVKGRFTISRDNPKNTLFLQMSSLRAEDTAVYYCVRVKVLYRGGMDVWGQ | |
| | GTTVTVSS | |
| 215 | EVQLLESGGGLVQPGGSLRLSCAASGFTLSSYAMHWVRQAPGKGLEYVSGISPSSAS | STSA-C27-78-78-VH |
| | TYYANSVKGRFTISRDNPKNTLFLQMSSLRAEDTAVYYCVRVKSKYRGGMDVWGQ | |
| | GTTVTVSS | |
| 216 | EVQLLESGGGLVQPGGSLRLSCAASGFTLSSYAMHWVRQAPGKGLEYVSGISGNSAS | STSA-C27-82-58-VH |
| | TYYANSVKGRFTISRDNPKNTLFLQMSSLRAEDTAVYYCVRVKLKYRGGMDVWGQG | |
| | TTVTVSS | |
| 217 | EVQLLESGGGLVQPGGSLRLSCAASGFTLSSYAMHWVRQAPGKGLEYVSGISHSGTS | STSA-C27-54-54-VH |
| | TYYANSVKGRFTISRDNPKNTLFLQMSSLRAEDTAVYYCVRVRVLYRGGMDVWGQ | |
| | GTTVTVSS | |
| 218 | EVQLLESGGGLVQPGGSLRLSCAASGFTLSSYAMHWVRQAPGKGLEYVSGISPSGVS | STSA-C27-36-36-VH |
| | TYYANSVKGRFTISRDNPKNTLFLQMSSLRAEDTAVYYCVRVKVKYRGGMDVWGQ | |
| | GTTVTVSS | |
| 219 | EVQLLESGGGLVQPGGSLRLSCAASGFTLSSYAMHWVRQAPGKGLEYVSGISSNGG | STSA-C27-53-53-VH |
| | STYYANSVKGRFTISRDNPKNTLFLQMSSLRAEDTAVYYCVRVFVRYRGGMDVWGQ | |
| | GTTVTVSS | |
| 220 | EVQLLESGGGLVQPGGSLRLSCAASGFTLSSYAMHWVRQAPGKGLEYVSGISPTSAS | STSA-C27-67-67-VH |
| | TYYANSVKGRFTISRDNPKNTLFLQMSSLRAEDTAVYYCVRVKGRYRGGMDVWGQ | |
| | GTTVTVSS | |
| 221 | EVQLLESGGGLVQPGGSLRLSCAASGFTLSSYAMHWVRQAPGKGLEYVSGISPTGGS | STSA-C27-55-55-VH |
| | TYYANSVKGRFTISRDNPKNTLFLQMSSLRAEDTAVYYCVRVKGRYRGGMDVWGQ | |
| | GTTVTVSS | |
| 222 | EVQLLESGGGLVQPGGSLRLSCAASGFTLSSYAMHWVRQAPGKGLEYVSGISHSGN | STSA-C27-59-59-VH |
| | STYYANSVKGRFTISRDNPKNTLFLQMSSLRAEDTAVYYCVRVKRRYRGGMDVWGQ | |
| | GTTVTVSS | |
| 223 | EVQLLESGGGLVQPGGSLRLSCAASGFTLSSYAMHWVRQAPGKGLEYVSGISPSSNS | STSA-C27-58-58-VH |
| | TYYANSVKGRFTISRDNPKNTLFLQMSSLRAEDTAVYYCVRVKVRYRGGMDVWGQ | |
| | GTTVTVSS | |
| 224 | EVQLLESGGGLVQPGGSLRLSCAASGFTLSSYAMHWVRQAPGKGLEYVSGISSSGSS | STSA-C27-52-52-VH |
| | TYYANSVKGRFTISRDNPKNTLFLQMSSLRAEDTAVYYCVRVKPAYRGGMDVWGQ | |
| | GTTVTVSS | |
| 225 | EVQLLESGGGLVQPGGSLRLSCAASGFTLSSYAMHWVRQAPGKGLEYVSGISYSSAS | STSA-C27-Y2-Y2-VH |
| | TYYANSVKGRFTISRDNPKNTLFLQMSSLRAEDTAVYYCVRVKVRYRGGMDVWGQ | |
| | GTTVTVSS | |
| 226 | ETTLTQSPDTLPLSPGDRASLSCRASQSVSSAYLAWYQQKPGQAPRLLIYGTSRRATG | STSA-C27-VL |
| | VPGRFSGSGSGTDFTLTISRLEPEDFAVYYCQLYGSSSVTFGQGTKLEIK | |
| 227 | EIVLTQSPGTLSLSPGERATLSCRASQGISSAYLAWYQQKPGQAPRLLIYGTSRRATGI | STSA-C27-6-33-VL |
| | PDRFSGSGSGTDFTLTISRLEPEDFAVYYCQLYGATSVTFGQGTKLEIK | |
| 228 | EIVLTQSPGTLSLSPGERATLSCRASQGISSAYLAWYQQKPGQAPRLLIYGTSRRATGI | STSA-C27-7-33-VL |
| | PDRFSGSGSGTDFTLTISRLEPEDFAVYYCQLYGATSVTFGQGTKLEIK | |
| 229 | EIVLTQSPGTLSLSPGERATLSCRASQSVSSAYLAWYQQKPGQAPRLLIYGTSRRATGI | STSA-C27-24-56-VL |
| | PDRFSGSGSGTDFTLTISRLEPEDFAVYYCQLYGASSVTFGQGTKLEIK | |
| 230 | EIVLTQSPGTLSLSPGERATLSCRASQSVSSAYLAWYQQKPGQAPRLLIYGTSRRATGI | STSA-C27-47-56-VL |
| | PDRFSGSGSGTDFTLTISRLEPEDFAVYYCQLYGASSVTFGQGTKLEIK | |
| 231 | EIVLTQSPGTLSLSPGERATLSCRASQGISSAYLAWYQQKPGQAPRLLIYGTSRRATGI | STSA-C27-33-33-VL |
| | PDRFSGSGSGTDFTLTISRLEPEDFAVYYCQLYGATSVTFGQGTKLEIK | |
| 232 | EIVLTQSPGTLSLSPGERATLSCRASQSVSSAYLAWYQQKPGQAPRLLIYGTSRRATGI | STSA-C27-56-56-VL |
| | PDRFSGSGSGTDFTLTISRLEPEDFAVYYCQLYGASSVTFGQGTKLEIK | |
| 233 | EIVLTQSPGTLSLSPGERATLSCRASQSISTAYLAWYQQKPGQAPRLLIYGTSRRATGIP | STSA-C27-78-78-VL |
| | DRFSGSGSGTDFTLTISRLEPEDFAVYYCQLYGASSVTFGQGTKLEIK | |
| 234 | EIVLTQSPGTLSLSPGERATLSCRASQDISSAYLAWYQQKPGQAPRLLIYGTSRRATGI | STSA-C27-82-58-VL |
| | PDRFSGSGSGTDFTLTISRLEPEDFAVYYCQLYGATSVTFGQGTKLEIK | |
| 235 | EIVLTQSPGTLSLSPGERATLSCRASQDVSSAYLAWYQQKPGQAPRLLIYGTSRRATGI | STSA-C27-54-54-VL |
| | PDRFSGSGSGTDFTLTISRLEPEDFAVYYCQLYGATSVTFGQGTKLEIK | |
| 236 | EIVLTQSPGTLSLSPGERATLSCRASQNISTAYLAWYQQKPGQAPRLLIYGTSRRATGI | STSA-C27-36-36-VL |
| | PDRFSGSGSGTDFTLTISRLEPEDFAVYYCQLYGATSVTFGQGTKLEIK | |
| 237 | EIVLTQSPGTLSLSPGERATLSCRASQDASNAYLAWYQQKPGQAPRLLIYGTSRRATG | STSA-C27-53-53-VL |
| | IPDRFSGSGSGTDFTLTISRLEPEDFAVYYCQLYGSSSVTFGQGTKLEIK | |
| 238 | EIVLTQSPGTLSLSPGERATLSCRASQGVSSAYLAWYQQKPGQAPRLLIYGTSRRATGI | STSA-C27-67-67-VL |
| | PDRFSGSGSGTDFTLTISRLEPEDFAVYYCQLYGRSSVTFGQGTKLEIK | |
| 239 | EIVLTQSPGTLSLSPGERATLSCRASQNISTAYLAWYQQKPGQAPRLLIYGTSRRATGI | STSA-C27-55-55-VL |
| | PDRFSGSGSGTDFTLTISRLEPEDFAVYYCQLYGTSSVTFGQGTKLEIK | |
| 240 | EIVLTQSPGTLSLSPGERATLSCRASQSVSTAYLAWYQQKPGQAPRLLIYGTSRRATGI | STSA-C27-59-59-VL |
| | PDRFSGSGSGTDFTLTISRLEPEDFAVYYCQLYGATSVTFGQGTKLEIK | |
| 241 | EIVLTQSPGTLSLSPGERATLSCRASQDISSAYLAWYQQKPGQAPRLLIYGTSRRATGI | STSA-C27-58-58-VL |
| | PDRFSGSGSGTDFTLTISRLEPEDFAVYYCQLYGATSVTFGQGTKLEIK | |
| 242 | EIVLTQSPGTLSLSPGERATLSCRASQGVSTAYLAWYQQKPGQAPRLLIYGTSRRATGI | STSA-C27-52-52-VL |
| | PDRFSGSGSGTDFTLTISRLEPEDFAVYYCQLYGATSVTFGQGTKLEIK | |
| 243 | EIVLPQSPGTLSLSPGERATLSCRASQGVSSAYLAWYQQKPGQAPRLLIYGTSRRATGI | STSA-C27-Y2-Y2-VL |
| | PDRFSGSGSGTDFTLTISRLEPEDFAVYYCQLYGSTSVTFGQGTKLEIK | |

What is claimed is:

1. A method for treating atopic dermatitis (AD) or improving an AD-associated parameter in a subject, the method comprising:
administering to a subject in need thereof one or more doses of an interleukin-4 receptor (IL-4R) antagonist, wherein the subject has moderate-to-severe or severe AD and is ≥6 months to <6 years of age, wherein the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

2. The method of claim 1, wherein the subject has moderate-to-severe or severe AD that is not adequately controlled by topical AD medications.

3. The method of claim 1 or 2, wherein the subject is inadequately responsive to treatment with a topical corticosteroid (TCS) of medium or higher potency.

4. The method of any one of claims 1 to 3, wherein the subject is a candidate for systemic AD therapy.

5. The method of any one of claims 1 to 3, wherein the subject previously was administered a systemic therapy for AD.

6. The method of any one of claims 1 to 5, wherein the subject is aged ≥6 months to <2 years.

7. The method of any one of claims 1 to 5, wherein the subject is aged ≥2 to <6 years.

8. The method of any one of claims 1 to 7, wherein the subject:
(i) has a baseline Investigator's Global Assessment (IGA) score ≥3;
(ii) has a baseline Eczema Area and Severity Index (EASI) score ≥16;
(iii) has a baseline Body Surface Area (BSA) affected by AD ≥10%; and/or
(iv) has a baseline weekly average score for maximum scratch/itch intensity ≥4.

9. The method of any one of claims 1 to 8, wherein the subject has a concurrent atopic or allergic condition selected from the group consisting of allergic rhinitis, asthma, food allergy, non-food allergy, allergic conjunctivitis, hives, chronic rhinosinusitis, nasal polyps, and eosinophilic esophagitis.

10. The method of claim 9, wherein the subject has a food allergy.

11. The method of any one of claims 1 to 9, wherein the subject has a baseline weight of ≥5 to <30 kg.

12. The method of claim 11, wherein the subject has a baseline weight of ≥5 to <15 kg.

13. The method of any one of claims 1 to 12, wherein:
for a subject with a baseline weight of ≥5 to <15 kg, the IL-4R antagonist is subcutaneously administered at a dose of 200 mg every four weeks (Q4W); and/or
for a subject with a baseline weight of ≥15 to <30 kg, the IL-4R antagonist is subcutaneously administered at a dose of 300 mg Q4W.

14. The method of claim 13, wherein the subject has a baseline weight ≥5 to <15 kg, and wherein the IL-4R antagonist is subcutaneously administered an initial dose of 200 mg followed by one or more subsequent doses of 200 mg Q4W.

15. The method of claim 13, wherein the subject has a baseline weight ≥15 to <30 kg, and wherein the IL-4R antagonist is subcutaneously administered an initial dose of 300 mg followed by one or more subsequent doses of 300 mg Q4W.

16. The method of any one of claims 1 to 15, wherein the IL-4R antagonist is administered for at least 16 weeks.

17. The method of any of claims 1 to 16, wherein the IL-4R antagonist is administered in combination with a topical AD medication.

18. The method of claim 17, wherein the topical AD medication is a low-potency TCS.

19. The method of claim 18, wherein treatment with the IL-4R antagonist:
results in an increase in the number of TCS medication-free days for the subject; and/or
results in a decrease in the weekly dose of TCS medication that is used by the subject.

20. The method of any one of claims 1 to 19, wherein treatment with the IL-4R antagonist decreases the need for a rescue treatment.

21. The method of any one of claims 1 to 20, wherein treatment with the IL-4R antagonist results in:
a reduction from baseline in IGA score to achieve an IGA score of 0 or 1 by Week 16 after administration of a first dose of the IL-4R antagonist; and/or
a reduction of at least 75% from baseline in an EASI score (EASI-75) by Week 16 after administration of a first dose of the IL-4R antagonist.

22. The method of any one of claims 1 to 20, wherein treatment with the IL-4R antagonist results in an improvement in an AD-associated parameter selected from the group consisting of:
a reduction of at least 50% from baseline in EASI score (EASI-50) by Week 1 after administration of a first dose of the IL-4R antagonist;
a reduction of at least 75% from baseline in EASI score (EASI-75) by Week 2 after administration of a first dose of the IL-4R antagonist;
a reduction of at least 90% from baseline in EASI score (EASI-90) by Week 4 after administration of a first dose of the IL-4R antagonist; and
a ≥4-point improvement in Pruritus NRS score by Week 3 after administration of a first dose of the IL-4R antagonist.

23. The method of any one of claims 1 to 20, wherein treatment with the IL-4R antagonist results in an improvement in an AD-associated parameter selected from the group consisting of:
a decrease of at least 50% in EASI from baseline to Week 16 after administration of a first dose of the IL-4R antagonist;
a decrease of at least 24% in percent BSA affected by AD from baseline to Week 16 after administration of a first dose of the IL-4R antagonist;
a decrease of at least 9 points in POEM score from baseline to Week 16 after administration of a first dose of the IL-4R antagonist;
a decrease of at least 38% in SCORAD score from baseline to Week 16 after administration of a first dose of the IL-4R antagonist;
an increase of at least 1.5 points in sleep quality NRS from baseline to Week 16 after administration of a first dose of the IL-4R antagonist;
a decrease of at least 3 points in skin pain NRS from baseline to Week 16 after administration of a first dose of the IL-4R antagonist;
a decrease of at least 7 points in CDLQI score from baseline to Week 16 after administration of a first dose of the IL-4R antagonist; and
a decrease of at least 8 points in IDQOL score from baseline to Week 16 after administration of a first dose of the IL-4R antagonist.

24. The method of any one of claims 1 to 23, wherein the AD-associated parameter is determined based on a caregiver-reported assessment.

25. The method of any one of claims 1 to 24, wherein treatment with the IL-4R antagonist prevents skin infection or reduces susceptibility to skin infection in the subject.

26. The method of any one of claims 1 to 25, wherein treatment with the IL-4R antagonist results in a reduction in the level of one or more type 2 inflammatory biomarkers in the subject relative to a baseline value.

27. The method of claim 26, wherein treatment with the IL-4R antagonist results in a reduction in the level of serum TARC and/or serum total IgE in the subject relative to a baseline value.

28. The method of any one of claims 1 to 27, wherein the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2.

29. The method of any one of claims 1 to 28, wherein the anti-IL-4R antibody comprises a heavy chain comprising the amino acid sequence of SEQ ID NO:9 and a light chain comprising the amino acid sequence of SEQ ID NO:10.

30. The method of any one of claims 1 to 29, wherein the IL-4R antagonist is dupilumab.

31. The method of any one of claims 1 to 30, wherein the IL-4R antagonist is contained in a container selected from the group consisting of a glass vial, a syringe, a pre-filled syringe, a pen delivery device, and an autoinjector.

32. The method of claim 31, wherein the IL-4R antagonist is contained in a pre-filled syringe.

33. The method of claim 32, wherein the pre-filled syringe is a single-dose pre-filled syringe.

34. The method of claim 31, wherein the IL-4R antagonist is contained in an autoinjector.

35. The method of claim 31, wherein the IL-4R antagonist is contained in a pen delivery device.

36. A therapeutic dosage form of a pharmaceutical composition comprising an IL-4R antagonist, wherein administration of the dosage form to a subject for at least 16 weeks provides a mean serum concentration of the IL-4R antagonist of about 110 mg/L.

37. The therapeutic dosage form of claim 36, wherein a therapeutic dose of 200 mg of the IL-4R antagonist is administered every four weeks.

38. The therapeutic dosage form of claim 36, wherein a therapeutic dose of 300 mg of the IL-4R antagonist is administered every four weeks.

39. The therapeutic dosage form of any one of claims 36 to 38, wherein the subject is 6 months to <6 years of age.

40. The therapeutic dosage form of any one of claims 36 to 39, wherein the IL-4R antagonist is an anti-IL-4R antibody or antigen-binding fragment thereof that comprises a heavy chain complementarity determining region (HCDR)1 comprising the amino acid sequence of SEQ ID NO:3, an HCDR2 comprising the amino acid sequence of SEQ ID NO:4, an HCDR3 comprising the amino acid sequence of SEQ ID NO:5, a light chain complementarity determining region (LCDR)1 comprising the amino acid sequence of SEQ ID NO:6, an LCDR2 comprising the amino acid sequence LGS, and an LCDR3 comprising the amino acid sequence of SEQ ID NO:8.

41. The therapeutic dosage form of claim 40, wherein the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2.


SEQUENCE LISTING (243 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 18 aa, synthetic construct
AKDRLSITIRPRYYGLDV

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 452 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, synthetic construct; identical to SEQ ID NO: 69

SEQ ID NO: 74 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, synthetic construct; identical to SEQ ID NO: 74

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, synthetic construct; identical to SEQ ID NO: 74

SEQ ID NO: 81 - protein, 116 aa, synthetic construct; identical to SEQ ID NO: 79

SEQ ID NO: 82 - protein, 111 aa, synthetic construct; identical to SEQ ID NO: 78

SEQ ID NO: 83 - protein, 116 aa, synthetic construct; identical to SEQ ID NO: 69

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, synthetic construct; identical to SEQ ID NO: 75

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, synthetic construct; identical to SEQ ID NO: 87

SEQ ID NO: 90 - protein, 111 aa, synthetic construct; identical to SEQ ID NO: 84

SEQ ID NO: 91 - protein, 116 aa, synthetic construct; identical to SEQ ID NO: 75

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, synthetic construct; identical to SEQ ID NO: 69

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, synthetic construct; identical to SEQ ID NO: 69

SEQ ID NO: 96 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, synthetic construct; identical to SEQ ID NO: 69

SEQ ID NO: 98 - protein, 111 aa, synthetic construct; identical to SEQ ID NO: 78

SEQ ID NO: 99 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, synthetic construct; identical to SEQ ID NO: 99

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, synthetic construct; identical to SEQ ID NO: 99

SEQ ID NO: 104 - protein, 111 aa, synthetic construct; identical to SEQ ID NO: 78

SEQ ID NO: 105 - protein, 116 aa, synthetic construct; identical to SEQ ID NO: 77

SEQ ID NO: 106 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, synthetic construct; identical to SEQ ID NO: 77

SEQ ID NO: 110 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, synthetic construct; identical to SEQ ID NO: 77

SEQ ID NO: 118 - protein, 111 aa, synthetic construct; identical to SEQ ID NO: 114

SEQ ID NO: 119 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, synthetic construct; identical to SEQ ID NO: 77

SEQ ID NO: 126 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, synthetic construct; identical to SEQ ID NO: 77

SEQ ID NO: 128 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, synthetic construct; identical to SEQ ID NO: 77

SEQ ID NO: 130 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, synthetic construct; identical to SEQ ID NO: 114

SEQ ID NO: 135 - protein, 116 aa, synthetic construct; identical to SEQ ID NO: 77

SEQ ID NO: 136 - protein, 111 aa, synthetic construct; identical to SEQ ID NO: 124

SEQ ID NO: 137 - protein, 116 aa, synthetic construct; identical to SEQ ID NO: 107

SEQ ID NO: 138 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, synthetic construct; identical to SEQ ID NO: 107

SEQ ID NO: 142 - protein, 111 aa, synthetic construct; identical to SEQ ID NO: 114

SEQ ID NO: 143 - protein, 116 aa, synthetic construct; identical to SEQ ID NO: 113

SEQ ID NO: 144 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, synthetic construct; identical to SEQ ID NO: 77

SEQ ID NO: 146 - protein, 111 aa, synthetic construct; identical to SEQ ID NO: 138

SEQ ID NO: 147 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, synthetic construct; identical to SEQ ID NO: 177

SEQ ID NO: 179 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, synthetic construct; identical to SEQ ID NO: 177

SEQ ID NO: 182 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, synthetic construct; identical to SEQ ID NO: 210

SEQ ID NO: 219 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, synthetic construct; identical to SEQ ID NO: 227

SEQ ID NO: 229 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, synthetic construct; identical to SEQ ID NO: 229

SEQ ID NO: 231 - protein, 108 aa, synthetic construct; identical to SEQ ID NO: 227

SEQ ID NO: 232 - protein, 108 aa, synthetic construct; identical to SEQ ID NO: 229

SEQ ID NO: 233 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, synthetic construct; identical to SEQ ID NO: 234

SEQ ID NO: 242 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK